(19) United States
(12) Patent Application Publication
(10) Pub. No.: US 2012/0077186 A1
(43) Pub. Date: Mar. 29, 2012

(54) USE OF CYSTEINE-DERIVED SUPPRESSOR TRNAS FOR NON-NATIVE AMINO ACID INCORPORATION

(75) Inventors: William Skach, Portland, OR (US); Zhongying Yang, Beaverton, OR (US); Soo Jung Kim, Portland, OR (US); Jacob Gubbens, Oegstgeest (NL); Arthur Johnson, College Station, TX (US)

(73) Assignee: Oregon Health & Science University

(21) Appl. No.: 13/101,993

(22) Filed: May 5, 2011

Related U.S. Application Data
(60) Provisional application No. 61/332,676, filed on May 7, 2010.

(51) Int. Cl. C12P 21/00; C12Q 1/68; C07H 21/02

(52) U.S. Cl. 435/6.1; 536/23.1; 435/68.1

(57) ABSTRACT

Disclosed herein are compositions and methods for incorporating non-native amino acids into a single polypeptide. In one example, an isolated modified suppressor tRNA is disclosed that includes the following: a modified tRNA^{cys}, wherein the tRNA^{cys} has been modified so that an anticodon of the tRNA is complementary to a stop codon; a cysteine amino acid residue is covalently linked to the modified tRNA^{cys} by aminoacylation generating a chemically reactive sulfhydryl side chain; and a detectable label is covalently linked to the sulfhydryl side chain. Methods for incorporating non-native amino acids into a single polypeptide utilizing the disclosed isolated modified suppressor tRNAs are also disclosed.

### CROSS REFERENCE TO RELATED APPLICATION

This application claims the benefit of and priority to U.S. Provisional Application No. 61/332,676 filed on May 7, 2010 which is incorporated herein by reference in its entirety.

### ACKNOWLEDGMENT OF GOVERNMENT SUPPORT

This invention was made with government support under Contract Nos. R01GM53457, DK51818, GM53457 and R01DK51818 awarded by National Institutes of Health. The government has certain rights in the invention.

### FIELD OF THE DISCLOSURE

This disclosure concerns compositions and methods for incorporating non-native amino acids into a polypeptide.

### BACKGROUND

Site-specific incorporation of non-natural amino acids is a tool to manipulate proteins for structural and functional studies, or to create proteins with new properties. This is usually accomplished by one of two methods: labeling presynthesized proteins with probes at reactive side chains such as cysteine or lysine residues, or incorporating probes into nascent proteins as they are being synthesized in the presence of a modified aminoacyl-tRNA (aa-tRNA). The former method generally requires extensive mutagenesis to remove redundant labeling sites and/or purification to eliminate labeled contaminants. In contrast, co-translational incorporation using modified aa-tRNAs and a mRNA containing a unique cognate codon allows diverse probes to be positioned at virtually any site with minimal perturbation of the protein sequence. The latter approach most commonly utilizes an amber suppressor aa-tRNA that recognizes a unique nonsense (UAG) codon. However, a variety of synthetic and engineered aa-tRNAs, including those that recognize four-base codons, have also been developed for this purpose.

To enable co-translational incorporation, the non-native amino acid must be attached to tRNA with high efficiency, either by using an engineered orthogonal aa-tRNA synthetase (aaRS) that recognizes both the modified amino acid and tRNA, or by chemical coupling to presynthesized tRNAs. While expression of an aaRS-tRNA pair enables probe incorporation in intact cells, generating suitable aaRS enzymes is labor intensive for higher eukaryotes and often limited to specific probe structures. aa-tRNAs containing a modified amino acid can also be generated in vitro by chemical acylation of a probe to a synthetic dinucleotide and subsequent ligation to the 3′-end of a truncated tRNA. Alternatively, coupling probes to enzymatically aminoacylated tRNAs is technically straightforward, and requires only that the tRNA is recognized by an aaRS and that the coupled amino acid contains a chemically reactive side chain such as a free amine (lysine) or a sulfhydryl group (cysteine). Enzymatic aminoacylation in vitro provides the opportunity to create radiolabeled modified aa-tRNAs and accurately quantify probe incorporation.

The above approaches have enabled incorporation of photoactive cross-linkers, azides/alkynes for ‘click’-chemistry, photocaged residues, spin labels, and fluorescent probes to measure binding affinities, protein structure and environment in higher eukaryotic expression systems. Unfortunately, concomitant incorporation of two different normative amino acids at defined locations in the same protein has proven much more difficult. To date, this has been achieved using an amber suppressor tRNA in combination with a lysyl tRNA, a four-base anticodon tRNA and a suppressor tRNA, and two different four-base anticodon tRNAs. However, each of these approaches requires mutagenesis to remove alternative incorporation sites, and the added modified aa-tRNAs must compete with endogenous aa-tRNAs.

Like amber suppressors, opal and ochre tRNAs are usually derived from a native scaffold in which the anticodon and/or adjacent bases have been mutated to optimize pairing with a UGA or UAA codon, respectively. In this manner, tRNA^{Phe}-derived opal and ochre tRNAs have been used to incorporate nitrophenylalanine in rabbit reticulocyte lysate (RRL), and opal suppressors derived from tRNA^{Gln} or tRNA^{Trp} have been used to incorporate 5-hydroxytryptophan and 5-F-tryptophan, respectively, in mammalian cells. Ochre suppressors derived from E. coli supF tRNA and suppressors of all three nonsense codons derived from E. coli tRNA^{Gln} have also been used to incorporate unmodified tyrosine and glutamine residues in mammalian cells, respectively. However, such efforts have been limited by the ability of aaRS's to recognize suppressor tRNAs and/or modified amino acids, and low readthrough levels at their cognate codons. To date, concurrent suppression of two sequential nonsense codons has been extremely low (<2.5% readthrough) and has been achieved only using natural amino acids.

### SUMMARY

Amber suppressor tRNAs are used to incorporate non-natural amino acids into proteins to serve as probes of structure, environment, and function. However, this approach is limited for many reasons, including, not permitting multiple probes to be simultaneously incorporated at different locations in the same protein without other modification. Disclosed herein are amber, opal, and ochre suppressor tRNAs derived from various organisms, including E. coli and yeast tRNA^{Cys}, that incorporate a chemically modified cysteine residue with high selectivity at cognate UAG, UGA, and UAA stop codons. These suppressor tRNAs enable readthrough at each of their cognate stop codons and allow sequential incorporation of one or more non-natural amino acids in a eukaryotic translation system with an efficiency approximately ten-fold higher than reported with previous technologies. These methods establish a versatile means to incorporate multiple non-natural amino acids at defined sites within a single protein that can serve multiple functions, such as probes of structure, environment, and function. Also disclosed herein is the use of suppressor tRNAs with an RNA aptamer that inhibits eukaryotic translation termination (release) factors eRF1 and eRF3. The resulting readthough efficiencies in a translation system were far superior to previous reports for opal and ochre suppressor tRNAs carrying non-native amino acids. The disclosed system greatly expands the ability to incorporate non-native amino acids and allows incorporation of multiple probes in the same protein at levels far more efficiently than previously reported systems.

As such, disclosed herein are isolated modified suppressor tRNAs. In some examples, an isolated modified suppressor tRNA is a cysteine-derived suppressor tRNA (tRNA^{cys}) that has been modified so that an anticodon of the tRNA is complementary to a stop codon, a cysteine amino acid residue is covalently linked to the modified tRNA^{cys} by aminoacylation generating a chemically reactive sulfhydryl side chain and a detectable label is covalently linked to the sulfhydryl side chain. In some examples, the stop codon is an amber (UAG), an opal (UGA) or an ochre (UAA) stop codon. In some examples, the detectable label functions as an aminoacyl-tRNA stabilizing molecule. In some examples, the detectable label is a fluorescent group, a phosphorescent group, a photoaffinity label, or a photo-caged group, a crosslinking agent, a polymer, a cytotoxic molecule, a saccharide, a heavy metal-binding element, a spin label, a heavy atom, a redox group, an infrared probe, a keto group, an azide group, or an alkyne group. Exemplary fluorescent groups include, but are not limited to, 7-nitrobenz-2-oxa-1,3-diazol (NBD) or 3-(bromomethyl)-2,5,6-trimethyl-1H,7H-pyrazolo(1,2-α)pyrazole-1,7-dione (MBB). In some examples, the detectable label is covalently linked to the sulfhydryl side chain by an iodoacetamide and maleimide ester derivative. The modified tRNA can be eukaryotic tRNA, such as a yeast tRNA or human tRNA or prokaryotic tRNA, such as an E. coli tRNA.

Also disclosed are methods of incorporating at least one non-natural amino acid into a single polypeptide. In some embodiments, these methods include contacting a template mRNA capable of in vitro translation containing at least one amber (UAG), an opal (UGA) or an ochre (UAA) stop codon with at least one of the disclosed isolated modified suppressor tRNA and at least one RNA aptamer in a cell-free in vitro translation system capable of in vitro translation under conditions sufficient such that at least one non-natural amino acid is incorporated into the single polypeptide at the site of translation of the amber, opal or ochre codon.

In some embodiments, the method is a method of incorporating at least two non-natural amino acids into a single polypeptide wherein the template mRNA contains at least two amber, opal, ochre or a combination thereof stop codons.

Also disclosed are kits including at least one of the disclosed isolated modified suppressor tRNAs and at least one RNA aptamer.

The foregoing and other features of the disclosure will become more apparent from the following detailed description of several embodiments, which proceeds with reference to the accompanying figures.

### BRIEF DESCRIPTION OF THE DRAWINGS

FIGS. 1A-1C are schematics illustrating an exemplary construct, AQP4.P, including mutations which allowed non-natural amino acids to be incorporated into a single polypeptide. Leucine 44 of an AQP4.P construct containing the first 46 amino acids of human AQP4, a Val-Thr linker, and amino acids 88-299 of bovine prolactin, was mutated to cysteine (TGC) or an amber (TAG), opal (TGA), or ochre (TAA) stop codon (A). Where the cysteine codon was used for incorporation, cysteine 88 of prolactin (residue 49 in this construct) was mutated to alanine. For dual probe incorporation, amber and opal stop codons were placed at residues Leu44 and His68, respectively. Where indicated, mRNA was truncated at codon 183 (B) or codon 98 (C) to generate constructs AQP4-P(183) and AQP4-P(98), respectively. Full length plasmids encode TAA at codon 191 and TGA at codon 200. Residues are numbered sequentially from Met1. Because prolactin contains multiple cysteine residues near its C-terminus, incorporation was quantified using constructs terminated at residue 98. For SDS-PAGE analysis of nonsense codon readthrough, full-length constructs (190 amino acids) were used, unless indicated otherwise. Readthrough of the final TAA codon would result in the addition of nine amino acids until an opal codon is reached.

FIG. 1D is a schematic of an exemplary in vitro translation system and in particular, the incorporation of two non-natural amino acids into a single polypeptide. It is contemplated that the template mRNA can have multiple stop codons at any site within the mRNA so that the tRNA is still able to bind and translation can occur. In some examples, the mRNA has a stop codon inserted into the first half of the mRNA or the second half of the template mRNA sequence. In other examples, one or more stop codons are inserted in a first third, a second third or the last third of the mRNA template sequence. In some examples, one or more stop codons is incorporated in the first quarter, the second quarter, the third quarter or the fourth quarter or in combinations thereof of the mRNA template sequence.

FIGS. 2A-2D illustrate the incorporation and stability of Cys-tRNAs in RRL and WG translation systems. Yeast, E. coli, or human tRNA^{Cys}s, and an E. coli tRNA^{amb(Lys)} were transcribed in vitro and enzymatically charged with [¹⁴C]Cys or [¹⁴C]Lys, respectively. FIG. 2A is a graph illustrating amino acid incorporation when tRNAs were added to RRL (grey bars) or WG (white bars) translation reactions programmed with AQP4.P mRNA truncated at codon 98 and containing a unique cysteine codon (UGC) or amber codon (UAG) at residue 44 (FIG. 1). Incorporation into nascent protein was measured by liquid scintillation counting of hot acid-precipitable counts. Results show mean+/−SEM (n≧3). (FIGS. 2B-2D are graphs illustrating deaminoacylation of Lys-tRNA^{amb}(), and yeast (▴), E. coli (▪), or human (▾) Cys-tRNA^{Cys} following incubation in a mock translation reaction containing RRL (FIG. 2B), WG (FIG. 2C), or buffer alone (FIG. 2D). At times indicated, aa-tRNA was precipitated in cold TCA and analyzed by scintillation counting. Counts obtained at t=0 were used as reference.

FIGS. 3A-3D illustrate the incorporation and stability of NBD-Cys-tRNAs in RRL and WG translation systems. FIG. 3A is a bar graph of aa-tRNAs described in FIGS. 2A-2D labeled with NBD and added to RRL and WG translation reactions programmed with similarly truncated AQP4.P mRNA or buffer, as indicated. Incorporation into protein at a unique amber or cysteine codons in RRL (grey bars) and WG (white bars) was measured as in FIG. 2A. Results show mean+/−SEM (n≧3). FIGS. 3B-3D are bar graphs illustrating deacylation of NBD-labeled aa-tRNAs (Lys-tRNA^{amb}(), and yeast (▴), E. coli (▪), or human (▾) Cys-tRNA^{Cys}) as function of incubation time as assayed as described in FIG. 2A.

FIG. 4A is a bar graph illustrating amino acid incorporation in which WG translation of AQP4.P truncated at amino acid 98 was carried out in WG for 1 hour, and incorporation of ¹⁴C-labeled Lys or Cys (white bars) and their NBD-labeled derivatives (grey bars) into translated protein was measured by liquid scintillation counting of hot acid-precipitable ¹⁴C counts.

FIG. 4B is a bar graph illustrating amino acid incorporation in which translation was carried out as in FIG. 4A, although incorporation of the NBD- or MBB-labeled amino acids was measured in the absence (white bars) or presence (grey bars) of 1 μM RNA aptamer. Results show mean+/−SEM (n≧3).

FIGS. 5A-5D illustrate translational readthrough by tRNA^{Cys}-derived suppressor tRNAs at amber, opal, or ochre codons. mRNA encoding full length AQP4.P with a unique amber (FIG. 5A), opal (FIG. 5B) or ochre (FIG. 5C) codon at position 44 (indicated at bottom of gel) was transcribed from supercoiled plasmid and translated in the presence of corresponding suppressor tRNA (indicated above gel). Translation was carried out in WG the presence of tran[³⁵S]-label with or without aptamer, and products analyzed by SDS-PAGE and phosphor imaging. Translational readthrough of suppressor tRNAs at UAG, UGA, and UAA codons in the presence of aptamer was analyzed by SDS-PAGE. Identity of the stop codon at residue 44 is indicated below the gel and the added tRNA is shown above the gel in FIG. 5D. (−) indicates protein terminated at codon 44, (*) and downward arrows indicate full-length protein.

FIGS. 6A-6E illustrate concomitant incorporation of two modified amino acids at cognate amber and opal stop codons. (FIG. 6A) AQP4.P mRNA transcribed from plasmid DNA and translated in the presence of Tran[³⁵S]-label, aptamer, and εNBD-[³H]Lys-tRNA^{amb}, yeast MBB-[¹⁴C]Cys-tRNA^{opl}, and/or yeast NBD-[¹⁴C]Cys-tRNA^{opl} as indicated. Products were analyzed by SDS-PAGE and phosphor imaging. Locations of UAG and UGA codons are indicated below gels. Products that terminate at the stop codon at residue 44 are marked by (−). Polypeptides that read through the stop codon at residue 44 or both stop codons are marked by (*) and (**), respectively. Concurrent readthrough efficiency of both stop codons was 16%, compared to WT protein (compare lanes 1 and 12). (FIG. 6B) mRNAs containing indicated stop codons were truncated at residue 183, translated in WG for 1 hour, and ribosome-nascent chain complexes were pelleted, RNAse treated, and analyzed by SDS-PAGE. Gel was scanned for NBD fluorescence as described in Methods. (FIG. 6C) Translation was performed as in panel a, except that mRNA was truncated at codon 98. Samples were RNAse treated before SDS-PAGE. Note that polypeptides terminated at residue 68 and 98 contain 2 versus 4 methionines, respectively. Concurrent readthrough efficiency, based on phosphorimaging, at both codons was 28% (compare lanes 1 and 12, **). (FIGS. 6D-6E) NBD-[³H]Lys-tRNA^{amb} and yeast MBB-[¹⁴C]Cys-tRNA^{opl} were added either individually (FIG. 6D) or together (FIG. 6E) to a translation reaction containing apt-12 and programmed with mRNA truncated at codon 98. Incorporation efficiency was measured as hot-acid precipitable counts. Values represent the mean of two studies.

### SEQUENCE LISTING

The nucleic acid and amino sequences listed herein are shown using standard letter abbreviations for nucleotide bases and amino acids, as defined in 37 C.F.R. 1.822. Only one strand of each nucleic acid sequence is shown, but the complementary strand is understood as included by any reference to the displayed strand; stop anticodons are in bold lettering. All Genbank Accession Numbers are incorporated by reference for the sequence available on May 7, 2010.

The Sequence Listing is submitted as an ASCII text file, Annex C/St.25 text file, created on May 2, 2011, 12.9 KB, which is incorporated by reference herein.

In the accompanying sequence listing:

SEQ ID NO: 1 is a nucleic acid sequence for
E coli tRNA^{Cys}
(GGCGCGTTAACAAAGCGGTTATGTAGCGGATTGCAAATCCGTCT
AGTCCGGTTCGACTCCGGAACGCGCCTCCA).
SEQ ID NO: 2 is a nucleic acid sequence for
E coli tRNA^{Cysoch}
(GGCGCGTTAACAAAGCGGTTATGTAGCGGATTTTAAATCCGTCT
AGTCCGGTTCGACTCCGGAACGCGCCTCCA).
SEQ ID NO: 3 is a nucleic acid sequence for
E coli tRNA^{Cysopl}
(GGCGCGTTAACAAAGCGGTTATGTAGCGGATTTCAAATCCGTCT
AGTCCGGTTCGACTCCGGAACGCGCCTCCA).
SEQ ID NO: 4 is a nucleic acid sequence for
Yeast tRNA^{Cys}
(GCTCGTATGGCGCAGTGGTAGCGCAGCAGATTGCAAATCTGTTG
GTCCTTAGTTCGATCCTGAGTGCGAGCTCCA).
SEQ ID NO: 5 is a nucleic acid sequence for
Yeast tRNA^{Cysamb}
(GCTCGTATGGCGCAGTGGTAGCGCAGCAGATTCTAAATCTGTTG
GTCCTTAGTTCGATCCTGAGTGCGAGCTCCA).
SEQ ID NO: 6 is a nucleic acid sequence for
Yeast tRNA^{Cysaopl}
(GCTCGTATGGCGCAGTGGTAGCGCAGCAGATTTCAAATCTGTTG
GTCCTTAGTTCGATCCTGAGTGCGAGCTCCA).
SEQ ID NO: 7 is a nucleic acid sequence for
Yeast tRNA^{Cysoch}
(GCTCGTATGGCGCAGTGGTAGCGCAGCAGATTTTAAATCTGTTG
GTCCTTAGTTCGATCCTGAGTGCGAGCTCCA).
SEQ ID NO: 8 is a nucleic acid sequence for
Human tRNA^{Cys}
(GGGGGTATAGCTCAGGGGTAGAGCATTTGACTGCAGATCAAGAG
GTCCCTGGTTCGAATCCAGGTGCCCCCTCCA).
SEQ ID NO: 9 is an amino acid sequence for
E coli cysteine tRNA synthethase
(MLKIFNTLTRQKEEFKPIHAGEVGMYVCGITVYDLCHIGHGRTF
VAFDVVARYLRFLGYKLKYVRNITDIDDKIIKRANENGESFVAMV
DRMIAEMHKDFDALNILRPDMEPRATHHIAEIIELTEQLIAKGHA
YVADNGDVMFDVPTDPTYGVLSRQDLDQLQAGARVDVVDDKRNPM
DFVLWKMSKEGEPSWPSPWGAGRPGWHIECSAMNCKQLGNHFDIH
GGGSDLMFPHHENEIAQSTCAHDGQYVNYWMHSGMVMVDREKMSK
SLGNFFTVRDVLKYYDAETVRYFLMSGHYRSQLNYSEENLKQARA
ALERLYTALRGTDKTVAPAGGEAFEARFIEAMDDDFNTPEAYSVL
FDMAREVNRLKAEDMAAANAMASHLRKLSAVLGLLEQEPEAFLQS
GAQADDSEVAEIEALIQQRLDARKAKDWAAADAARDRLNEMGIVL
EDGPQGTTWRRK;
GenBank Accession No. M59381 which is hereby
incorporated by reference in its entirety).
SEQ ID NO: 10 is an amino acid sequence for
human cysteine tRNA synthethase
(MADSSGQQGKGRRVQPQWSPPAGTQPCRLHLYNSLTRNKEVFIP
QDGKKVTWYCCGPTVYDASHMGHARSYISFDILRRVLKDYFKFDV
FYCMNITDIDDKIIKRARQNHLFEQYREKRPEAAQLLEDVQAALK
PFSVKLNETTDPDKKQMLERIQHAVQLATEPLEKAVQSRLTGEEV
NSCVEVLLEEAKDLLSDWLDSTLGCDVTDNSIFSKLPKFWEGDFH
RDMEALNVLPPDVLTRVSEYVPEIVNFVQKIVDNGYGYVSNGSVY
FDTAKFASSEKHSYGKLVPEAVGDQKALQEGEGDLSISADRLSEK
RSPNDFALWKASKPGEPSWPCPWGKGRPGWHIECSAMAGTLLGAS
MDIHGGGFDLRFPHHDNELAQSEAYFENDCWVRYFLHTGHLTIAG
CKMSKSLKNFITIKDALKKHSARQLRLAFLMHSWKDTLDYSSNTM
ESALQYEKFLNEFFLNVKDILRAPVDITGQFEKWGEEEAELNKNF
YDKKTAIHKALCDNVDTRTVMEEMRALVSQCNLYMAARKAVRKRP
NQALLENIALYLTHMLKIFGAVEEDSSLGFPVGGPGTSLSLEATV
MPYLQVLSEFREGVRKIAREQKVPEILQLSDALRDNILPELGVRF
EDHEGLPTVVKLVDRNTLLKEREEKRRVEEEKRKKKEEAARRKQE
QEAAKLAKMKIPPSEMFLSETDKYSKFDENVSMVCPHMTWRAKSS
AKGKPRS*RSSSRLRRSSTRNICRWPRMEASSEGAQD;
GenBank Accession No. AF288207 is hereby
incorporated by reference in its entirety).
SEQ ID NO: 11 is a nucleic acid sequence for
Aptamer 34
(GGGAGCTCAGAATAAACGCTCAACATCACCGTACGCCGGGCAAC
TGGCGCTGATTCGACATGAGACACGGATCCTGC).
SEQ ID NO: 12 is a nucleic acid sequence for
Aptamer 12
(GGGAGCTCAGAATAAACGCTCAAGTACCTGAAAATGGGAAGCAG
AGCGAGCCTTTCGACATGAGACACGGATCCTGC).

### DETAILED DESCRIPTION OF SEVERAL EMBODIMENTS

### I. Overview of Several Embodiments

As such, disclosed herein are isolated modified suppressor tRNAs. In some examples, an isolated modified suppressor tRNA is a cysteine-derived suppressor tRNA (tRNA^{cys}) that has been modified so that an anticodon of the tRNA is complementary to a stop codon, a cysteine amino acid residue is covalently linked to the modified tRNA^{cys} by aminoacylation generating a chemically reactive sulfhydryl side chain and a detectable label is covalently linked to the sulfhydryl side chain. In some examples, the stop codon is an amber (UAG), an opal (UGA) or an ochre (UAA) stop codon. In some examples, the detectable label functions as an aminoacyl-tRNA stabilizing molecule. In some examples, the detectable label is a fluorescent group, a phosphorescent group, a photoaffinity label, or a photo-caged group, a crosslinking agent, a polymer, a cytotoxic molecule, a saccharide, a heavy metal-binding element, a spin label, a heavy atom, a redox group, an infrared probe, a keto group, an azide group, or an alkyne group. Exemplary fluorescent groups include, but are not limited to, 7-nitrobenz-2-oxa-1,3-diazol (NBD) or 3-(bromomethyl)-2,5,6-trimethyl-1H,7H-pyrazolo(1,2-α)pyrazole-1,7-dione (MBB). In some examples, the detectable is covalently linked to the sulfhydryl side chain by an iodoacetamide and maleimide ester derivative.

In some embodiments, the modified tRNA is eukaryotic tRNA, such as a yeast tRNA or human tRNA. In some embodiments, the modified tRNA is prokaryotic tRNA, such as an E. coli tRNA.

Also disclosed are kits including at least one of the disclosed isolated modified suppressor tRNAs and at least one RNA aptamer. In some embodiments, at least one RNA aptamer is an RNA aptamer which suppresses one or more translation termination release factors. In some embodiments, the RNA aptamer is an RNA aptamer capable of suppressing translation termination release factor 1 (eRF1), translation termination release factor 3 (eRF3) or a combination thereof. In some embodiments, the at least one RNA aptamer is RNA aptamer 12 or RNA aptamer 34.

Also disclosed are methods of incorporating at least one non-natural amino acid into a single polypeptide. In some embodiments, these methods include contacting a template mRNA capable of in vitro translation containing at least one amber (UAG), an opal (UGA) or an ochre (UAA) stop codon with at least one of the disclosed isolated modified suppressor tRNA and at least one RNA aptamer in a cell-free in vitro translation system capable of in vitro translation under conditions sufficient such that at least one non-natural amino acid is incorporated into the single polypeptide at the at the site of translation of the amber, opal or ochre codon.

In some embodiments, the method is a method of incorporating at least two non-natural amino acids into a single polypeptide wherein the template mRNA contains at least two amber, opal, ochre stop codons or a combination thereof. In some embodiments, the at least one RNA aptamer suppresses one or more translation termination release factors, such as eRF1 and eRF3.

In some embodiments, the translation system is a Wheat Germ translation system.

In some embodiments, the translation system is a reticulocyte lysate translation system.

In some embodiments, the method further includes preparing at least one isolated modified suppressor tRNAs. For example, preparing the at least one isolated modified suppressor tRNAs includes combining the modified suppressor tRNA^{cys} with cysteine, an E. coli extract and a purified recombinant aminoacyl-tRNA synthetase under conditions sufficient for the cysteine amino acid residue to be covalently linked to the modified tRNA to generate a modified suppressor tRNA with a cysteine including a chemically reactive sulfhydryl side chain; and combining a detectable label with the modified suppressor tRNA so that the detectable label is covalently linked to the chemically reactive sulfhydryl side chain.

### II. Abbreviations and Terms

Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: A Comprehensive Desk Reference, published by VCH Publishers, Inc., 1995 (ISBN 1-56081-569-8).

The singular terms “a,” “an,” and “the” include plural referents unless context clearly indicates otherwise. “Comprising” means “including.” “Comprising A or B” means “including A,” “including B” or “including A and B.” It is further to be understood that all base sizes or amino acid sizes, and all molecular weight or molecular mass values, given for nucleic acids or peptides are approximate, and are provided for description.

Suitable methods and materials for the practice or testing of the disclosure are described below. However, the provided materials, methods, and examples are illustrative only and are not intended to be limiting. Accordingly, except as otherwise noted, the methods and techniques of the present disclosure can be performed according to methods and materials similar or equivalent to those described and/or according to conventional methods well known in the art and as described in various general and more specific references that are cited and discussed throughout the present specification (see, for instance, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999).

i. Abbreviations
aaRS: tRNA synthetase
aa-tRNA: aminoacyl-tRNA
amb: amber
cys: cysteine
MBB: 3-(bromomethyl)-2,5,6-trimethyl-1H,7H-pyrazolo(1,2-α)pyrazole-1,7-dione
NBD: 7-nitrobenz-2-oxa-1,3-diazol
och: ochre
opl: opal
RRL: rabbit reticulocyte lysate
mRNA: messenger RNA
tRNA: transfer RNA

ii. Terms

In order to facilitate review of the various embodiments of the disclosure, the following explanations of specific terms are provided:

Aptamer: Oligonucleic acid or peptide molecules that bind to a specific target molecule. Aptamers are usually created by selecting them from a large random sequence pool, but natural aptamers also exist in riboswitches. Aptamers can be used for both basic research and clinical purposes as macromolecular drugs. Aptamers can be combined with ribozymes to self-cleave in the presence of their target molecule. DNA or RNA aptamers typically include short strands of oligonucleotides.

Nucleic acid sequences for aptamers are publicly available. For example, exemplary aptamer sequences are available within the Aptamer Database found on the World Wide Web at domain name aptamer.icmb.utexas.edu, all of which are incorporated by reference as provided on May 7, 2010. In some examples, an aptamer has a nucleic acid sequence provided by SEQ ID NOS: 11 or 12, or a variant thereof, such as a nucleic acid sequence with at least 50% sequence identity, for example at least about 60%, such as at least about 70%, at least about 80%, at least about 85%, at least about 90%, at least about 95%, or at least about 98%, including 61%, 62%, 63,%, 64%, 65%, 67%, 68%, 69%, 70%, 71%, 72%, 73%, 74%, 75%, 76%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NOS: 11 or 12. In some examples, the RNA aptamer is an RNA aptamer which suppresses one or more translation termination release factors, such as translation termination release factor 1 (eRF1), translation termination release factor 3 (eRF3) or a combination thereof. In one particular example, an RNA aptamer is RNA aptamer 12 or RNA aptamer 34 (see for example, SEQ ID NO: 12 or 11, respectively).

Bacteria: Unicellular microorganisms belonging to the Kingdom Procarya. Unlike eukaryotic cells, bacterial cells do not contain a nucleus and rarely harbour membrane-bound organelles. As used herein, both Archaea and Eubacteria are encompassed by the terms “prokaryote” and “bacteria.” Examples of Eubacteria include, but are not limited to Escherichia coli, Thermus thermophilus and Bacillus stearothermophilus. Example of Archaea include Methanococcus jannaschii, Methanosarcina mazei, Methanobacterium thermoautotrophicum, Methanococcus maripaludis, Methanopyrus kandleri, Halobacterium such as Haloferax volcanii and Halobacterium species NRC-i, Archaeoglobus fulgidus, Pyrococcus fit riosus, Pyrococcus horikoshii, Pyrobaculum aerophilum, Pyrococcus abyssi, Sulfolobus solfataricus, Sulfolobus tokodaii, Aeuropyrum pernix, Thermoplasma acidophilum, and Thermoplasma volcanium.

Complementarity and percentage complementarity: Molecules with complementary nucleic acids form a stable duplex or triplex when the strands bind, (hybridize), to each other by forming Watson-Crick, Hoogsteen or reverse Hoogsteen base pairs. Stable binding occurs when an oligonucleotide molecule remains detectably bound to a target nucleic acid sequence (such as an ovarian endothelial cell tumor-associated molecule) under the required conditions.

Complementarity is the degree to which bases in one nucleic acid strand base pair with the bases in a second nucleic acid strand. Complementarity is conveniently described by percentage, that is, the proportion of nucleotides that form base pairs between two strands or within a specific region or domain of two strands. For example, if 10 nucleotides of a 15-nucleotide oligonucleotide form base pairs with a targeted region of a DNA molecule, that oligonucleotide is said to have 66.67% complementarity to the region of DNA targeted.

In the present disclosure, “sufficient complementarity” means that a sufficient number of base pairs exist between an oligonucleotide molecule and a target nucleic acid sequence to achieve detectable binding. When expressed or measured by percentage of base pairs formed, the percentage complementarity that fulfills this goal can range from as little as about 50% complementarity to full (100%) complementary. In general, sufficient complementarity is at least about 50%, for example at least about 75% complementarity, at least about 90% complementarity, at least about 95% complementarity, at least about 98% complementarity, or even at least about 100% complementarity.

A thorough treatment of the qualitative and quantitative considerations involved in establishing binding conditions that allow one skilled in the art to design appropriate oligonucleotides for use under the desired conditions is provided by Beltz et al. Methods Enzymol. 100:266-285, 1983, and by Sambrook et al. (ed.), Molecular Cloning: A Laboratory Manual, 2nd ed., vol. 1-3, Cold Spring Harbor Laboratory Press, Cold Spring Harbor, N.Y., 1989.

Contacting: Placement in direct physical association, including both a solid and liquid form. In one example, contacting occurs in vitro, for example, with isolated cells.

Covalently linked: Refers to a covalent linkage between atoms by the formation of a covalent bond characterized by the sharing of pairs of electrons between atoms. In one example, a cysteine amino acid residue is covalently linked to the modified tRNA to generate a modified suppressor tRNA with a cysteine including a chemically reactive sulfhydryl side chain. In some examples, a detectable label is covalently linked to the chemically reactive sulfhydryl side chain.

Cysteine (Cys): An α-amino acid with the chemical formula HO₂CCH(NH₂)CH₂SH and its position within a protein is encoded by codons in either DNA or RNA sequence, such as codons UGU and UGC. The side chain on cysteine which often participates in enzymatic reactions, serving as a nucleophile. The thiol is susceptible to oxidization to give the disulfide derivative cystine, which serves a structural role in many proteins.

Encode: Any process whereby the information in a polymeric macromolecule or sequence is used to direct the production of a second molecule or sequence that is different from the first molecule or sequence. As used herein, the term is construed broadly, and can have a variety of applications. In some aspects, the term “encode” describes the process of semi-conservative DNA replication, where one strand of a double-stranded DNA molecule is used as a template to encode a newly synthesized complementary sister strand by a DNA-dependent DNA polymerase.

In another aspect, the term “encode” refers to any process whereby the information in one molecule is used to direct the production of a second molecule that has a different chemical nature from the first molecule. For example, a DNA molecule can encode an RNA molecule (for instance, by the process of transcription incorporating a DNA-dependent RNA polymerase enzyme). Also, an RNA molecule can encode a peptide, as in the process of translation. When used to describe the process of translation, the term “encode” also extends to the triplet codon that encodes an amino acid. In some aspects, an RNA molecule can encode a DNA molecule, for instance, by the process of reverse transcription incorporating an RNA-dependent DNA polymerase. In another aspect, a DNA molecule can encode a peptide, where it is understood that “encode” as used in that case incorporates both the processes of transcription and translation.

Eukaryote: Organisms belonging to the Kingdom Eucarya. Eukaryotes are generally distinguishable from prokaryotes by their typically multicellular organization (but not exclusively multicellular, for example, yeast), the presence of a membrane-bound nucleus and other membrane-bound organelles, linear genetic material (for instance, linear chromosomes), the absence of operons, the presence of introns, message capping and poly-A mRNA, and other biochemical characteristics known in the art, such as a distinguishing ribosomal structure. Eukaryotic organisms include, for example, animals (for instance, mammals, insects, reptiles, birds, etc.), ciliates, plants (for instance, monocots, dicots, algae, etc.), fungi, yeasts, flagellates, microsporidia, and protists. A eukaryotic cell is one from a eukaryotic organism, for instance a human cell or a yeast cell.

Hybridization: Oligonucleotides and their analogs hybridize to one another by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence.

“Specifically hybridizable” and “specifically complementary” are terms that indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11.

For purposes of the present disclosure, “stringent conditions” encompass conditions under which hybridization will only occur if there is less than 25% mismatch between the hybridization molecule and the target sequence. “Stringent conditions” can be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize.

In particular embodiments, stringent conditions are hybridization at 65° C. in 6×SSC, 5×Denhardt's solution, 0.5% SDS and 100 μg sheared salmon testes DNA, followed by 15-30 minute sequential washes at 65° C. in 2×SSC, 0.5% SDS, followed by 1×SSC, 0.5% SDS and finally 0.2×SSC, 0.5% SDS.

In vitro translation: Translation of a protein from an RNA template in a cell-free system that has the components necessary for translation of a protein. In some instances, in vitro translation is coupled with in vitro transcription, such as a DNA template can be used. Examples of cell-free systems in which in vitro transcription or translation can occur are a wheat germ translation system, reticulocyte lysate translation system (such as rabbit reticulocyte lysate translation), E. coli translation system and commercially available systems, such as those sold by 5 PRIME (Germany).

Isolated: An “isolated” biological component (such as a nucleic acid molecule, peptide, or cell) has been purified away from other biological components in a mixed sample (such as a cell extract). For example, an “isolated” peptide or nucleic acid molecule is a peptide or nucleic acid molecule that has been separated from the other components of a cell in which the peptide or nucleic acid molecule was present (such as an expression host cell for a recombinant peptide or nucleic acid molecule). Nucleic acids, peptides and proteins that have been isolated include nucleic acids and proteins purified by standard purification methods, such as chromatography, for example high performance liquid chromatography (HPLC) and the like. The term also embraces nucleic acids, peptides, and proteins prepared by recombinant expression in a host cell as well as chemically synthesized peptide and nucleic acids. It is understood that the term “isolated” does not imply that the biological component is free of trace contamination, and can include molecules that are at least 50% isolated, such as at least 50%, 55%, 60%, 65%, 70%, 75%, 80%, 85%, 90%, 95%, 98%, 99%, or even 100% isolated.

Label: A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes (for example ¹⁴C, ³²P, ¹²⁵I, ³H isotopes and the like). In some examples a tRNA or protein, is labeled with a radioactive isotope, such as ¹⁴C, ³²P, ¹²⁵I, ³H isotope. In some examples, a fluorescent probe, such as NBD, MBB or like molecules are coupled to a disclosed tRNA. For example, in some embodiments such probe stabilizes the tRNA molecule. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998), Harlow & Lane (Antibodies, A Laboratory Manual, Cold Spring Harbor Publications, New York, 1988).

Mammalian cell: A cell from a mammal, the class of vertebrate animals characterized by the production of milk in females for the nourishment of young, from mammary glands present on most species; the presence of hair or fur; specialized teeth; three small bones within the ear; the presence of a neocortex region in the brain; and endothermic or “warm-blooded” bodies, and, in most cases, the existence of a placenta in the ontogeny. The brain regulates endothermic and circulatory systems, including a four-chambered heart. Mammals encompass approximately 5,800 species (including humans), distributed in about 1,200 genera, 152 families and up to forty-six orders, though this varies with the classification scheme.

Non-natural amino acid or Unnatural amino Acid: Any amino acid, modified amino acid, modified amino acid, and/or amino acid analogue other than selenocysteine and/or pyrrolysine and the following twenty genetically encoded alpha-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine. The generic structure of an alpha-amino acid is illustrated by Formula I: H₂NCH(R)COOH.

A unnatural amino acid typically is any structure having Formula I wherein the R group is any substituent other than one used in the twenty natural amino acids. See for instance, Biochemistry by L. Stryer, 31(1 ed. 1988), Freeman and Company, New York, for structures of the twenty natural amino acids. Unnatural amino acids also can be naturally occurring compounds other than the twenty alpha-amino acids above.

Specific, non-limiting examples of unnatural amino acids include p-ethylthiocarbonyl-L-phenylalanine, p-(3-oxobutanoyl)-L-phenylalanine, 1,5-dansyl-alanine, 7-amino-coumarin amino acid, 7-hydroxy-coumarin amino acid, nitrobenzyl-serine, O-(2-nitrobenzyl)-L-tyrosine, p-carboxymethyl-L-phenylalanine, p-cyano-L-phenylalanine, m-cyano-L-phenylalanine, biphenylalanine, 3-amino-L-tyrosine, bipyridyl alanine, p-(2-amino-1-hydroxyethyl)-L-phenylalanine, p-isopropylthiocarbonyl-L-phenylalanine, 3-nitro-L-tyrosine and p-nitro-L-phenyl alanine. Also, a p-propargyloxyphenylalanine, a 3,4-dihydroxy-L-phenyalanine (DIHP), a 3,4,6-trihydroxy-L-phen ylalanine, a 3,4,5-trihydroxy-L-phenylalanine, 4-nitro-phenylalanine, a p-acetyl-L-phenylalanine, O-methyl-L-tyrosine, an L-3-(2-naphthyl)alanine, a 3-methyl-phenylalanine, an O-4-allyl-L-tyrosine, a 4-propyl-L-tyrosine, a 3-nitro-tyrosine, a 3-thiol-tyrosine, a to-O-acetyl-GlcNAc-serine, an L-Dopa, a fluorinated phenylalanine, an isopropyl-L-phenylalanine, a p-azi do-L-phenyl alanine, a p-acyl-L-phenylalanifle, a p-benzoyl-L-phenylalanine, an L-phosphoserifle, a phosphonoserine, a phosphonotyrosine, a p-iodo-phenylalanine, a p-bromophenylalanine, a p-amino-L-phenylalanine, and an isopropyl-L-phenylalanine, and the like. See also, Published International Application WO 2004/094593 and Wang & Schultz (2005) Angewandte Chemie mt. Ed., 44(1):34-66, the content of which is incorporated by reference in its entirety.

In some unnatural amino acids, R in Formula I optionally includes an alkyl-, aryl-, acyl-, hydrazine, cyano-, halo-, hydrazide, alkenyl, ether, borate, boronate, phospho, phosphono, phosphine, enone, imine, ester, hydroxylamine, or amine group or the like, or any combination thereof. Other unnatural amino acids of interest include, but are not limited to, amino acids comprising a crosslinking amino acid, photoactivatable crosslinking amino acids, spin-labeled amino acids, fluorescent amino acids, metal binding amino acids, metal-containing amino acids, radioactive amino acids, amino acids with novel functional groups, amino acids that covalently or noncovalently interact with other molecules, photocaged and/or photoisomerizable amino acids, photoaffinity labeled amino acids, biotin or biotin-analogue containing amino acids, polymer-containing amino acids, cytotoxic molecule-containing amino acids, saccharide-containing amino acids, heavy metal-binding element-containing amino acids, amino acids containing a heavy atom, amino acids containing a redox group, amino acids containing an infrared probe, amino acids containing an azide group, amino acids containing an alkyne group, keto containing amino acids, glycosylated amino acids, a saccharide moiety attached to the amino acid side chain, amino acids comprising polyethylene glycol or polyether, heavy atom substituted amino acids, chemically cleavable or photocleavable amino acids, amino acids with an elongated side chain as compared to natural amino acids (for instance, polyethers or long chain hydrocarbons, for instance, greater than about 5, greater than about 10 carbons, etc.), carbon-linked sugar-containing amino acids, amino thioacid containing amino acids, and amino acids containing one or more toxic moieties.

In addition to unnatural amino acids that contain novel side chains, unnatural amino acids also can optionally include modified backbone structures, for instance, as illustrated by the structures of Formulas II and III:

ZCH(R)C(X)YH II

H₂NC(R¹)(R²)CO₂H III

wherein Z typically includes OH, NH₂, SH, NH—R², or S—R²; X and Y, which can be the same or different, typically include S or O, and R¹ and R², which are optionally the same or different, are typically selected from the same list of constituents for the R group described above for the unnatural amino acids having Formula I as well as hydrogen. For example, unnatural amino optionally include substitutions in the amino or carboxyl group as illustrated by Formulas II and III. Unnatural amino acids of this type include, but are not limited to, α-hydroxy acids, α-thioacids α-aminothiocarboxylates, for instance, with side chains corresponding to the common twenty natural amino acids or unnatural side chains. In some embodiments, the unnatural amino acids are used in the L-configuration. However, the disclosure is not limited to the use of L-configuration unnatural amino acids, and D-enantiomers of these unnatural amino acids also can be used.

Nucleic acid molecule: A polymeric form of nucleotides, which can include both sense and anti-sense strands of RNA, cDNA, genomic DNA, and synthetic forms and mixed polymers of the above. A nucleotide refers to a ribonucleotide, deoxynucleotide or a modified form of either type of nucleotide. A “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A nucleic acid molecule can include either or both naturally occurring and modified nucleotides linked together by naturally occurring and/or non-naturally occurring nucleotide linkages.

Nucleic acid molecules can be modified chemically or biochemically or can contain non-natural or derivatized nucleotide bases, as will be readily appreciated by those of skill in the art. Such modifications include, for example, labels, methylation, substitution of one or more of the naturally occurring nucleotides with an analog, internucleotide modifications, such as uncharged linkages (for example, methyl phosphonates, phosphotriesters, phosphoramidates, carbamates, etc.), charged linkages (for example, phosphorothioates, phosphorodithioates, etc.), pendent moieties (for example, peptides), intercalators (for example, acridine, psoralen, etc.), chelators, alkylators, and modified linkages (for example, alpha anomeric nucleic acids, etc.). The term “nucleic acid molecule” also includes any topological conformation, including single-stranded, double-stranded, partially duplexed, triplexed, hairpinned, circular and padlocked conformations.

Plasmid: A DNA molecule separate from chromosomal DNA and capable of autonomous replication. It is typically circular and double-stranded, and can naturally occur in bacteria, and sometimes in eukaryotic organisms (for instance, the 2-micrometre-ring in Saccharomyces cerevisiae). The size of plasmids can vary from about 1 to over 400 kilobase pairs. Plasmids often contain genes or gene cassettes that confer a selective advantage to the bacterium (or other cell) harboring them, such as the ability to make the bacterium (or other cell) antibiotic resistant.

Plasmids contain at least one DNA sequence that serves as an origin of replication, which enables the plasmid DNA to be duplicated independently from the chromosomal DNA. The chromosomes of most bacteria are circular, but linear plasmids are also known.

Plasmids used in genetic engineering are referred to as vectors. They can be used to transfer genes from one organism to another, and typically contain a genetic marker conferring a phenotype that can be selected for or against. Most also contain a polylinker or multiple cloning site, which is a short region containing several commonly used restriction sites allowing the easy insertion of DNA fragments at this location. Specific, non-limiting examples of plasmids include pCLHF, pCLNCX (Imgenex), pCLHF-GFP-TAG, pSUPER (OligoEngine), pEYCUA-YRS, pBluescript II KS (Stratagene), pcDNA3 (Invitrogen).

Peptide: Any compound composed of amino acids, amino acid analogs, chemically bound together. Peptide as used herein includes oligomers of amino acids, amino acid analog, or small and large peptides, including polypeptides or proteins. A peptide is any chain of amino acids, regardless of length or post-translational modification (such as glycosylation or phosphorylation). In one example, a peptide is two or more amino acids joined by a peptide bond. Typically, a peptide consists of fewer than fifty amino acids; for example, consisting of approximately 7 to approximately 40 amino acids, consisting of approximately 7 to approximately 30 amino acids, consisting of approximately 7 to approximately 20 amino acids.

“Peptide” applies to amino acid polymers to naturally occurring amino acid polymers and non-naturally occurring amino acid polymer as well as in which one or more amino acid residue is a non-natural amino acid, for example a artificial chemical mimetic of a corresponding naturally occurring amino acid.

A “polypeptide” is a polymer in which the monomers are amino acid residues which are joined together through amide bonds. When the amino acids are alpha-amino acids, either the L-optical isomer or the D-optical isomer can be used. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced. The term “residue” or “amino acid residue” includes reference to an amino acid that is incorporated into a protein, polypeptide, or peptide.

As used herein, the term “polypeptide fragment” refers to a portion of a polypeptide which exhibits at least one useful epitope or functional domain. Polypeptide fragments contemplated herein include all fragments of a polypeptide that retain a particular desired activity of the polypeptide. Biologically functional fragments can vary in size and will depend on the polypeptide of interest.

The term “soluble” refers to a form of a polypeptide that is not inserted into a cell membrane.

Conservative amino acid substitutions are those substitutions that, when made, least interfere with the properties of the original protein, that is, the structure and especially the function of the protein is conserved and not significantly changed by such substitutions. Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

Prokaryote: Organisms belonging to the Kingdom Monera (also termed Procarya). Prokaryotic organisms are generally distinguishable from eukaryotes by their unicellular organization, asexual reproduction by budding or fission, the lack of a membrane-bound nucleus or other membrane-bound organelles, a circular chromosome, the presence of operons, the absence of introns, message capping and poly-A mRNA, and other biochemical characteristics, such as a distinguishing ribosomal structure. The Prokarya include subkingdoms Eubacteria and Archaea (sometimes termed “Archaebacteria”). Cyanobacteria (the blue green algae) and mycoplasma are sometimes given separate classifications under the Kingdom Monera.

Purified: The term “purified” does not require absolute purity; rather, it is intended as a relative term. Thus, for example, a purified protein preparation is one in which the protein referred to is more pure than the protein in its natural environment within a cell. For example, a preparation of a protein is purified such that the protein represents at least 50% of the total protein content of the preparation. Similarly, a purified tRNA preparation is one in which the tRNA is more pure than in an environment including a complex mixture of tRNAs.

Readthrough: The continuation of translation when a mutation has converted a normal stop codon into one encoding an amino acid. This results in extension of the polypeptide chain until the next stop codon is reached, producing a so-called readthrough protein. Readthrough can also occur in transcription, of DNA beyond a normal stop signal, or terminator sequence, due to failure of RNA polymerase to recognize the signal.

RNA: A long chain polymer which is a complementary and modified form of the DNA in a cell. The term RNA generally implies the total RNA content of a cell, including messenger RNA (mRNA), ribosomal RNA, and transfer RNA (tRNA), and is generally derived from the cytoplasm of a cell. RNA is distinct from DNA in that it is only single-stranded and contains a uracil base while DNA contains a thymine.

Sequence identity: The similarity between two nucleic acid sequences or between two amino acid sequences is expressed in terms of the level of sequence identity shared between the sequences. Sequence identity is typically expressed in terms of percentage identity; the higher the percentage, the more similar the two sequences. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer.

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity.

When aligning short peptides (fewer than around 30 amino acids), the alignment is be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, 99% sequence identity. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least about 60%, 70%, 80%, 90%, 95%, 98%, or 99% sequence identity determined by this method. An alternative (and not necessarily cumulative) indication that two nucleic acid sequences are substantially identical is that the polypeptide which the first nucleic acid encodes is immunologically cross reactive with the polypeptide encoded by the second nucleic acid.

One of skill in the art will appreciate that the particular sequence identity ranges are provided for guidance only; it is possible that strongly significant homologs could be obtained that fall outside the ranges provided.

Substitution: The replacement of one thing with another. With reference to an amino acid in a polypeptide “substitution” means replacement of one amino acid with a different amino acid.

Suppression: To reduce the quality, amount, or strength of something. In one example, an RNA aptamer suppresses one or more translation termination release factors. For example, an RNA aptamer decreases or inhibits the activity of RNA aptamer 12 or 34, for example by at least 10%, 20%, 30%, 40%, 50%, 60%, 70%, 80%, 90%, 95% or 95% as compared to the response in the absence of the RNA apatmer. Such decreases can be measured using the methods disclosed herein.

Suppressor tRNA: A suppressor tRNA is a tRNA that alters the reading of a messenger RNA (mRNA) in a given translation system, for instance, by providing a mechanism for incorporating an amino acid into a peptide chain in response to a selector codon. For example, a suppressor tRNA can read through, for instance, a stop codon (for instance, an amber, ocher or opal codon), a four-base codon, a missense codon, a frameshift codon, or a rare codon. Stop codons include, for example, the ochre codon (UAA), amber codon (UAG), and opal codon (UGA). Exemplary modified suppressor tRNAs include a modified tRNA^{cys}, wherein the tRNA^{cys} has been modified so that an anticodon of the tRNA is complementary to a stop codon; a cysteine amino acid residue covalently linked to the modified tRNA^{cys} by aminoacylation generating a chemically reactive sulfhydryl side chain; and a detectable label covalently linked to the sulfhydryl side chain. For example, the modified tRNA^{cys} can be a tRNA^{cys} variants, fragments, homologs or fusion sequences that retain the ability to be transfer the amino acid cysteine, to a growing polypeptide chain. In certain examples, a modified suppressor tRNA includes a tRNA^{cys} with at least 50% sequence identity, for example at least 60%, 70%, 80%, 85%, 90%, 95%, or 98%, including 61%, 62%, 63,%, 64%, 65%, 67%, 68%, 69%, 70%, 71%, 72%, 73%, 74%, 75%, 76%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NOS: 1-8; in such variants the anticodons are not varied.

Translation termination release factor: A protein that allows for the termination of translation by recognizing the termination codon or stop codon in a mRNA sequence. During translation of mRNA, most codons are recognized by aminoacyl-tRNAs because they are adhered to specific amino acids corresponding to each tRNA's anticodon. Prokaryotic translation termination is mediated by three release factors: RF1 RF2 and RF3. RF1 recognizes the termination codons UAA and UAG. RF2 recognizes UAA and UGA. RF3 is a GTP-binding protein that facilitates the binding of RF1 and RF2 to the ribosomal complex. Eukaryotic translation termination similarly involves two release factors: eRF1 and eRF3. eRF1 recognizes all three termination codons. eRF3 is a ribosome-dependent GTPase that helps eRF1 release the completed polypeptide. In some examples, an RNA aptamer suppresses the activity of one or more translation termination release factor.

Transfer RNA (tRNA): A small RNA chain (generally 73-93 nucleotides) that transfers a specific amino acid to a growing peptide chain at the ribosomal site of protein synthesis during translation. It has a 3′ terminal site for amino acid attachment. This covalent linkage is catalyzed by an aminoacyl tRNA synthetase. It also contains a three-base region called the anticodon that can base-pair to the corresponding three base codon region on mRNA. Each type of tRNA molecule can be attached to only one type of amino acid, but because the genetic code contains multiple codons that specify the same amino acid, tRNA molecules bearing different anticodons can also carry the same amino acid.

Transfer RNA has a primary structure, a secondary structure (usually visualized as the cloverleaf structure), and a tertiary structure (an L-shaped three-dimensional structure that allows the tRNA to fit into the P and A sites of the ribosome). The acceptor stem is a 7-bp stem made by the base pairing of the 5′-terminal nucleotide with the 3′-terminal nucleotide (which contains the CCA 3′-terminal group used to attach the amino acid). The acceptor stem can contain non-Watson-Crick base pairs. The CCA tail is a CCA sequence at the 3′ end of the tRNA molecule that is used for the recognition of tRNA by enzymes involved in translation. In prokaryotes, the CCA sequence is transcribed, whereas in eukaryotes, the CCA sequence is added during processing and therefore does not appear in the tRNA gene.

In one example, a tRNA includes a full-length wild-type (or native) sequence, as well as tRNA variants, fragments, homologs or fusion sequences that retain the ability to be transfer a specific active amino acid, such as a cysteine, to a growing polypeptide chain. In certain examples, a tRNA has at least 50% sequence identity, for example at least 60%, 70%, 80%, 85%, 90%, 95%, or 98%, including 61%, 62%, 63,%, 64%, 65%, 67%, 68%, 69%, 70%, 71%, 72%, 73%, 74%, 75%, 76%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NOS: 1-8; in such variants the anticodons are not varied.

An anticodon is a unit made up of three nucleotides that correspond to the three bases of the mRNA codon. Each tRNA contains a specific anticodon triplet sequence that can base-pair to one or more codons for an amino acid. For example, one codon for lysine is AAA; the anticodon of a lysine tRNA might be UUU. Some anticodons can pair with more than one codon due to a phenomenon known as wobble base pairing. Frequently, the first nucleotide of the anticodon is one of two not found on mRNA: inosine and pseudouridine, which can hydrogen bond to more than one base in the corresponding codon position. In the genetic code, it is common for a single amino acid to occupy all four third-position possibilities; for example, the amino acid glycine is coded for by the codon sequences GGU, GGC, GGA, and GGG. To provide a one-to-one correspondence between tRNA molecules and codons that specify amino acids, 61 tRNA molecules would be required per cell. However, many cells contain fewer than 61 types of tRNAs because the wobble base is capable of binding to several, though not necessarily all, of the codons that specify a particular amino acid.

Aminoacylation is the process of adding an aminoacyl group to a compound. It produces tRNA molecules with their CCA 3′ ends covalently linked to an amino acid. Each tRNA is aminoacylated (or charged) with a specific amino acid by an aminoacyl tRNA synthetase. There is normally a single aminoacyl tRNA synthetase for each amino acid, despite the fact that there can be more than one tRNA, and more than one anticodon, for an amino acid. Recognition of the appropriate tRNA by the synthetases is not mediated solely by the anticodon, and the acceptor stem often plays a role.

Amino acid sequences for aminoacyl-tRNA synthetases are publicly available. For example, GenBank Accession Nos.: M59381 and AF288207disclose E. coli and human tRNA synthetase sequences, all of which are incorporated by reference as provided by GenBank on May 7, 2010.

Under conditions sufficient for: A phrase that is used to describe any environment that permits the desired activity. In one example, the desired activity is the covalently linking a cysteine amino acid residue to a modified tRNA to generate a modified suppressor tRNA with a cysteine including a chemically reactive sulfhydryl side chain.

Vector: A nucleic acid molecule capable of transporting a non-vector nucleic acid sequence which has been introduced into the vector. One type of vector is a “plasmid,” which refers to a circular double-stranded DNA into which non-plasmid DNA segments can be ligated. Other vectors include cosmids, bacterial artificial chromosomes (BAC) and yeast artificial chromosomes (YAC). Another type of vector is a viral vector, wherein additional DNA segments can be ligated into all or part of the viral genome. Certain vectors are capable of autonomous replication in a host cell into which they are introduced (for example, vectors having a bacterial origin of replication replicate in bacteria hosts). Other vectors can be integrated into the genome of a host cell upon introduction into the host cell and are replicated along with the host genome. Some vectors contain expression control sequences (such as promoters) and are capable of directing the transcription of an expressible nucleic acid sequence that has been introduced into the vector. Such vectors are referred to as “expression vectors.” A vector can also include one or more selectable marker genes and/or genetic elements known in the art.

Yeast: A eukaryotic microorganism classified in the Kingdom Fungi, with about 1,500 species described. Most reproduce asexually by budding, although a few reproduce by binary fission. Yeasts generally are unicellular, although some species may become multicellular through the formation of a string of connected budding cells known as pseudohyphae, or false hyphae. Exemplary yeasts that can be used in the disclosed methods and kits include but are not limited to Saccharomyces cerevisiae, Candida albicans, Schizosaccharomyces pombe, and Saccharomycetales.

### III. Isolated Modified Suppressor tRNAs

Disclosed herein are modified suppressor tRNAs. In some examples, an isolated modified suppressor tRNA is a cysteine-derived suppressor tRNA (tRNA^{cys}) that has been modified so that an anticodon of the tRNA is complementary to a stop codon, a cysteine amino acid residue is covalently linked to the modified tRNA^{cys} by aminoacylation generating a chemically reactive sulfhydryl side chain and a detectable label is covalently linked to the sulfhydryl side chain. In some examples, the stop codon is an amber (UAG), an opal (UGA) or an ochre (UAA) stop codon.

In some embodiments, an isolated modified suppressor tRNA is disclosed. Exemplary isolated modified suppressor tRNAs include a modified tRNA^{cys}, a cysteine amino acid residue covalently linked to the modified tRNA^{cys} and a detectable label. In some examples, the tRNA^{cys} has been modified so that an anticodon of the tRNA is complementary to a stop codon, such as an amber (UAG), an opal (UGA) or an ochre (UAA) stop codon. In an example, the modified tRNA is a eukaryotic (such as a yeast tRNA or human tRNA or a prokaryotic tRNA such as an E. coli tRNA. For example, the modified tRNA^{cys} can be a tRNA^{cys} variants, fragments, homologs or fusion sequences that retain the ability to be transfer the amino acid cysteine, to a growing polypeptide chain. In certain examples, a modified suppressor tRNA includes a tRNA^{cys} with at least 50% sequence identity, for example at least about 60%, at least about 70%, at least about 80%, at least about 85%, at least about 90%, at least about 95%, or at least about 98%, including 61%, 62%, 63,%, 64%, 65%, 67%, 68%, 69%, 70%, 71%, 72%, 73%, 74%, 75%, 76%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NOS: 1-8; in such variants the anticodons to the respective stop codons are not varied. In additional examples, the modified tRNA includes an anti-codon that is complementary to a stop codon, such as an amber (UAG), an opal (UGA) or an ochre (UAA) stop codon and is able to bind to a mRNA template to suppress the mRNA stop codon and allow a probe of interest to be incorporated into the polypeptide chain.

In some examples, the cysteine amino acid within the isolated modified suppressor tRNA is covalently linked to the modified tRNA^{cys} by aminoacylation generating a chemically reactive sulfhydryl side chain. In an example, the detectable label within the modified suppressor tRNA is covalently linked to the sulfhydryl side chain, such as by an iodoacetamide and maladamide ester derivative. In some examples, the detectable label functions as an aminoacyl-tRNA stabilizing molecule. The detectable label can be any small molecule known to those of ordinary skill in the art that allows the molecule of interest to be measured, such as a fluorescent group, a phosphorescent group, a photoaffinity label, or a photo-caged group, a crosslinking agent, a polymer, a cytotoxic molecule, a saccharide, a heavy metal-binding element, a spin label, a heavy atom, a redox group, an infrared probe, a keto group, an azide group, or an alkyne group. In some examples, the detectable label is a fluorescent group, such as 7-nitrobenz-2-oxa-1,3-diazol (NBD) or 3-(bromomethyl)-2,5,6-trimethyl-1H,7H-pyrazolo(1,2-α)pyrazole-1,7-dione (MBB).

### IV. Methods of Incorporating Non-Natural Amino Acids

Also disclosed is a method of incorporating at least one non-natural amino acid into a single polypeptide. In an example, the method includes contacting a template mRNA capable of in vitro translation containing at least one amber (UAG), an opal (UGA) or an ochre (UAA) stop codon with at least one isolated modified suppressor tRNA and at least one RNA aptamer in a cell-free in vitro translation system capable of in vitro translation under conditions sufficient such that at least one non-natural amino acid is incorporated into the single polypeptide at the at the site of translation of the amber, opal or ochre codon. In one example, the method is used to incorporate at least two non-natural amino acids (such as 2-5, 10-20, 20-50, 50-100 non-natural amino acids, such as 3, 4, 5, 6, 7, 8, 9, 10, 20, 30, 40, 50, 70, 80 or more non-natural amino acids) into a single polypeptide wherein the template mRNA contains at least two amber, opal or ochre stop codons (such as 2-5, 10-20, 20-50, 50-100 stop codons, such as 3, 4, 5, 6, 7, 8, 9, 10, 20, 30, 40, 50, 70, 80 or more stop codons). Exemplary translation systems include any of those known to one of ordinary skill in the art, including, but not limited to a Wheat Germ translation system, a reticulocyte lysate translation system or an E. coli translation system and other commercially available translation systems such as those commercially available from 5-PRIME (Germany).

In some examples, the method further includes preparing the at least one isolated modified suppressor tRNAs. For example, preparing the at least one isolated modified suppressor tRNAs includes combining the modified suppressor tRNA^{cys} with cysteine, an E. coli extract and a purified recombinant aminoacyl-tRNA synthetase under conditions sufficient for the cysteine amino acid residue to be covalently linked to the modified tRNA to generate a modified suppressor tRNA with a cysteine including a chemically reactive sulfhydryl side chain. In one example, preparing the at least one isolated modified suppressor tRNAs includes combining a detectable label with a modified suppressor tRNA so that the detectable label is covalently linked to the chemically reactive sulfhydryl side chain.

### V. Kits

Kits are also a feature of this disclosure. For example, a kit including at least one isolated modified suppressor tRNA and at least one RNA aptamer for incorporating at least one non-natural amino acid into a single peptide are disclosed herein. In some examples, the kit includes a disclosed isolated modified suppressor tRNA and at least one RNA aptamer is an RNA aptamer which suppresses one or more translation termination release factors, such as translation termination release factor 1 (eRF1), translation termination release factor 3 (eRF3) or a combination thereof. In particular examples, the at least one RNA aptamer is RNA aptamer 12 or RNA aptamer 34. For example, the RNA aptamer 12 has an amino acid sequence set forth by SEQ ID NO: 12 or has at least 70%, at least 80%, at least 90%, at least 95%, or at least 98% sequence identity with the amino acid sequence of SEQ ID NO: 12. In other examples, the RNA aptamer 34 has an amino acid sequence set forth by SEQ ID NO: 11 or has at least 70%, at least 80%, at least 90%, at least 95%, or at least 98% sequence identity with the amino acid sequence of SEQ ID NO: 11. In additional examples, kits also include the necessary components to perform in vitro translation such as the components necessary for translation of a protein. For example, a kit includes a wheat germ translation system, reticulocyte lysate translation system (such as rabbit reticulocyte lysate translation), E. coli translation system in combination with one or more disclosed isolated modified suppressor tRNAs and RNA aptamers.

The following examples are provided to illustrate certain particular features and/or embodiments. These examples should not be construed to limit the disclosure to the particular features or embodiments described.

### EXAMPLES

### Example 1

This example provides the material and methods utilized to perform efficient in vitro incorporation of non-natural amino acids into proteins using tRNA^{Cys}-derived opal, ochre, and amber suppressor tRNAs.

Plasmids and transcription. Plasmid pSP64-MIWC2 (Shi et al., Biochemistry 34(26): 8250-8256, 1995), henceforth called AQP4.P to conform with new nomenclature, encodes the 46 N-terminal residues of AQP4, a Thr-Val linker (BsteII restriction site), and the 142 C-terminal residues (aa88-229) of bovine preprolactin (pPL). Codon Leu44 of AQP4-P was converted to TGC (Cys), TAG (amber), TGA (opal), or TAA (ochre) by PCR overlap extension (Ho et al., Gene 77(1): 51-59, 1989). Similarly, Cys49 was converted to alanine, and where indicated His68 was converted to TGA (opal). cDNA was truncated by PCR amplification using a 5′-oligonucleotide complementary to the pSP64 vector (bp 2757) and a 3′-oligonucleotide ending at codon 98 or 183, which converted the last translated codon to valine to increase peptidyl-tRNA bond stability. Plasmid DNA or truncated PCR product was transcribed in vitro using SP6 polymerase under standard conditions described elsewhere (Oberdorf et al., Cystic Fibrosis Methods and Protocols, Vol. 70 (ed. W. R. Skach), Humana Press, Inc. Totowa, N.J., 2002).

Plasmids encoding yeast tRNA^{cys} or E. coli tRNA^{amb(Lys)} are described elsewhere (Alder et al., Cell 134(3): 439-450, 2008; Flanagan et al., J. Biol. Chem. 278(20): 18628-18637, 2003). Plasmids encoding E. coli and human tRNA^{cys} and their corresponding aaRS were provided by Ya-Ming Hou. Except for human tRNA^{Cys}, all tRNA sequences together with the T7 promoter were ligated into the SP64 plasmid by using EcoRI and HindIII sites introduced by PCR. The cysteine anticodon was then converted to an amber, opal, or ochre anticodon by PCR overlap extension. For transcription, tRNA sequences and the T7 promoter were amplified by PCR, and the resulting DNA transcribed in vitro using T7 RNA polymerase. tRNAs were purified by FPLC using a MonoQ column as described (Flanagan et al., J. Biol. Chem. 278(20): 18628-18637, 2003).

RNA aptamer was generated from synthetic overlapping DNA oligonucleotides encoding aptamer, T7 promoter, and EcoRI and HindIII sites. PCR products were ligated into pSP64 and transcription and purification of RNAs was performed as for tRNAs.

tRNA aminoacylation and modification. tRNA aminoacylation and purification was performed as described (Alder et al., Cell 134(3): 439-450, 2008; Johnson et al., Nucl. Acid. Res. 8(18): 4185-4200, 1980) with the following modifications: For tRNA^{amb(Lys)}, reactions contained 100 mM HEPES (pH 8.0), 8 mM MgCl₂, 1 mM DTT, 4 mM ATP, 0.1 mM CTP, 50 A₂₆₀ units of purified tRNA^{amb(Lys)}, 25% (v/v) DMSO, 12 μM [¹⁴C]Lys (Sigma, St. Louis, Mo.) or [³H]Lys (GE Healthcare, Piscataway, N.J.), and 10% (v/v) S-100 E. coli enzyme extract (Johnson et al., Biochemistry 15(3): 569-575, 1976; Menninger et al., Biochemica et biophysica acta 217(2): 496-511, 1970; Alder et al., Cell 134(3): 439-450, 2008). The reaction was incubated at 37° C. for 90 min. For tRNA^{cys} and suppressor derivatives, the reactions contained 100 mM HEPES (pH 7.5), 10 mM MgCl₂, 10 mM DTT, 4 mM ATP, 0.1 mM CTP, 50 A₂₆₀ units of tRNA, 25% (v/v) DMSO, 5 μM [¹⁴C]cystine (Perkin Elmer, Waltham, Mass.), preincubated with DTT as described [Crowley, 1993 #15], and 10% (v/v) of S-100 E. coli enzyme extract. Where indicated, His₆-tagged E. coli or human Cys-aaRS was expressed in E. coli, purified as described (Liu et al., J. Mol. Bio. 367(4): 1063-1078, 2007 and Liu et al., Nat. Methods 4:239-244, 2007), and added to final concentrations of 125 μg/ml and 320 μg/ml, respectively, together with 6% (v/v) S-100 extract. The reaction was carried out at 37° C. for 45 min.

The ε-amino group of Lys-tRNA^{amb} was chemically modified with succinimidyl 6-(N-(7-nitrobenz-2-oxa-1,3-diazol-4-yl)amino)hexanoate (Invitrogen, Carlsbad, Calif.) as described (Crowley et al., Cell 73(6): 1101-1115, 1993). The thiol group of Cys-tRNAs was chemically modified with N,N′-dimethyl-N-(iodoacetyl)-N′-(7-nitrobenz-2-oxa-1,3-diazol-4-yl)ethylenediamine (Invitrogen) as described (Alder et al., Cell 134(3): 439-450, 2008). Alternatively, 3-(bromomethyl)-2,5,6-trimethyl-1H,7H-Pyrazolo(1,2-α)pyrazole-1,7-dione (Invitrogen) was coupled to Cys-tRNAs under the same conditions. All modified aa-tRNAs were purified by reversed phase-HPLC (Alder et al., Cell 134(3): 439-450, 2008).

In vitro translation. AQP4-P fusion protein was translated at 24° C. for 1 hour in 10 μL cell-free translation reactions containing 20% (v/v) of crude transcription reaction and 40% (v/v) hemin-supplemented RRL (Oberdorf et al., Cystic Fibrosis Methods and Protocols, Vol. 70 (ed. W. R. Skach), Humana Press, Inc. Totowa, N.J., or 20% (v/v) WG (Erickson et al., Methods. Enzym. 96: 38-50, 1983). For undesalted RRL, 10 mM Tris acetate (pH 7.5), 100 mM KOAc, and 2 mM MgOAc was added, while desalted WG was adjusted to a final concentration 20 mM HEPES/KOH (pH 7.5), 140 mM KOAc, and 3 mM MgOAc. Translations also contained: 2 mM DTT, 0.4 mM spermidine, 1 mM ATP, 1 mM GTP, 12 mM creatine phosphate, 40 μM of all 20 amino acids, 40 μg/ml creatine kinase, 0.2 U/μL RNAse inhibitor, and 1 μM of tRNA, as indicated. Where Cys-tRNA^{Cys} was added, cysteine was omitted from the translation mixture. To detect translation products by SDS-PAGE, 1 μCi/μL Tran[³⁵S]-label (MP Biomedicals, Solon, Ohio) was added instead of methionine. Where indicated, 1 μM of RNA aptamer was added. For stability assays, a mock transcription mixture lacking DNA template was used.

Analysis of translation products. To quantify incorporation of ¹⁴C-labeled or ³H-labeled amino acids, 4 μL of translation reaction was incubated in 96 μL 1M NaOH, 2% (v/v) H₂O₂ at 37° C. for 10 min, and protein was precipitated in 10% (w/v) TCA at 85° C. (Daniel et al., J. Biol. Chem. 283(3): 20864-20873, 2008). Precipitate was collected by filtration (Durapore HVLP02500, Millipore, Billerica, Mass.) and subjected to liquid scintillation counting (Beckmann 6500, Fullerton, Calif.). Background incorporation was subtracted using a construct lacking the codon for incorporation. To distinguish between isotopes, samples that contained only ³H or ¹⁴C were used to set energy windows. After background subtraction, the lower energy window detected 100% of the ³H counts (counting efficiency of 9.6% cpm/dpm) and 33% of the ¹⁴C counts (counting efficiency of 85% cpm/dpm), whereas 67% of the ¹⁴C counts were detected in the higher energy window. Since all samples were treated similarly, quenching of the samples and therefore the energy distribution of the counts remained constant. By subtracting the relative contribution of ¹⁴C counts from the lower energy window and adding to the higher energy window net ³H and ¹⁴C counts were determined for samples containing both radioisotopes. To measure aminoacyl-tRNA stability, aliquots were taken after 0, 1, 5, 15, and 60 minutes incubations and analyzed as above, except that bleaching was not performed, and precipitation was carried out on ice for 30 min to prevent aminoacyl-tRNA hydrolysis.

For SDS-PAGE, 1 μL of ³⁵S-containing translation was separated on 12-17% (w/v) polacrylamide gels. Gels were dried, exposed on a phosphor imaging screen (Eastman Kodak, Rochester, N.Y.), and analyzed using a Bio-Rad FX molecular imager with Quantity One software (Bio-Rad, Hercules, Calif.). For in-gel fluorescence detection, ribosome-nascent chain complexes were pelleted at 350,000×g for 1 hour at 4° C. and resuspended in 10 mM TRIS/HCl (pH 8.0), 0.1% (w/v) SDS. Peptidyl tRNA was digested with 0.05 mg/ml Rnase A at 24° C. for 15 minutes and samples were analyzed by SDS-PAGE. In-gel fluorescence was detected using a Fuji film (Tokyo, Japan) FLA-5000 imager equipped with a 473 nm laser and a >510 nm high-pass filter.

### Example 2

This example demonstrates efficient in vitro incorporation of non-natural amino acids into proteins using tRNA^{Cys}-derived opal, ochre, and amber suppressor tRNAs.

Studies were first performed that demonstrated efficient aminoacylation and stability of synthetic Cys-tRNAs. To establish tRNA^{cys} as a viable platform for developing suppressor tRNAs, E. coli, yeast, and human tRNA^{cys} were characterized for their aminoacylation efficiency, aa-tRNA stability, and ability to incorporate of a modified cysteine residue in two eukaryotic cell free translation systems, wheat germ (WG) and RRL. tRNAs were synthesized by in vitro transcription and aminoacylated with [¹⁴C]Cys using an E. coli cytosolic extract. Although initial charging efficiencies were modest, aminoacylation was improved upon addition of purified recombinant E. coli and/or human cysteine aaRS (Table 1).

TABLE 1
Aminoacylation of tRNAs.

| | | Aminoacylation^{a} | | | | |
|---|---|---|---|---|---|---|
| | | (pmol/A₂₆₀ U tRNA used) | | | | |
| | | S-100 extract | | | | |
| | | + | + | + | − | − |
| | | Cys aaRS | | | | |
| Species | Codon | — | E. coli | Human | E. coli | Human |

Scaffold tRNA

| | | | |
|---|---|---|---|
| Lys | E. coli | Amber | 405 |
| | | (UAG) | |

Cys-tRNAs

| | | | | | | | |
|---|---|---|---|---|---|---|---|
| Cys | Yeast | Cys | 284 | 491 | 293 | 417 | 280 |
| | | (UGC) | | | | | |
| Cys | E. coli | Cys | 172 | 480 | 321 | 435 | 241 |
| | | (UGC) | | | | | |
| Cys | Human | Cys | 111 | 510 | 317 | 404 | 278 |
| | | (UGC) | | | | | |

Suppressor tRNAs

| | | | | | | | |
|---|---|---|---|---|---|---|---|
| Cys | Yeast | Amber | 5 | 13 | 433 | 32 | 362 |
| | | (UAG) | | | | | |
| Cys | Yeast | Opal | 17 | 29 | 453 | 7 | 352 |
| | | (UGA) | | | | | |
| Cys | Yeast | Ochre | 15 | 24 | 448 | 8 | 324 |
| | | (UAA) | | | | | |
| Cys | E. coli | Opal | 21 | 405 | 398 | 319 | 312 |
| | | (UGA) | | | | | |
| Cys | E. coli | Ochre | 3 | 454 | 474 | 313 | 368 |
| | | (UAA) | | | | | |

^{a}Yield of aminoacylation using [¹⁴C]Lys for Lys-tRNA and [¹⁴C]Cys for Cys-tRNA was calculated by quantifying the amount of radiolabeled tRNA by liquid scintillation counting per A₂₆₀ unit tRNA used in the reaction. Values are the average of two independent studies.

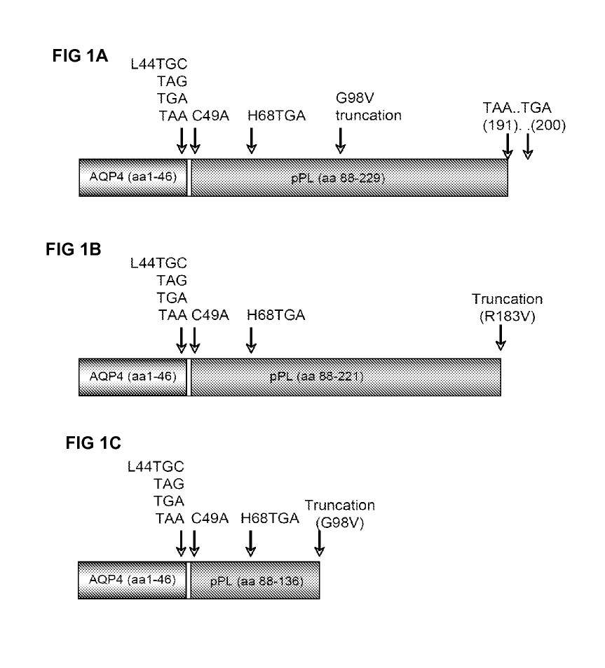

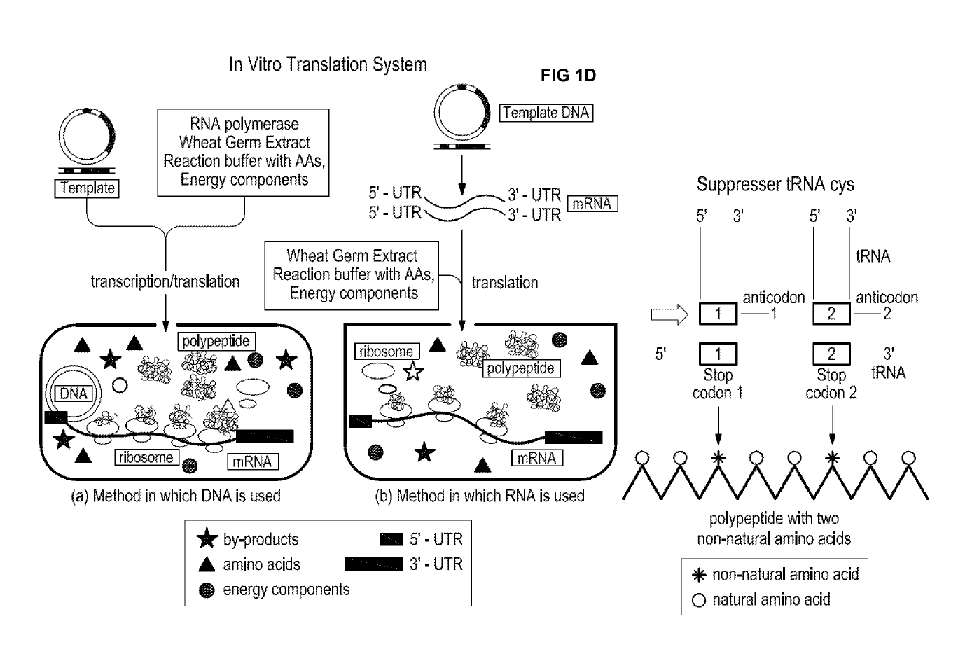

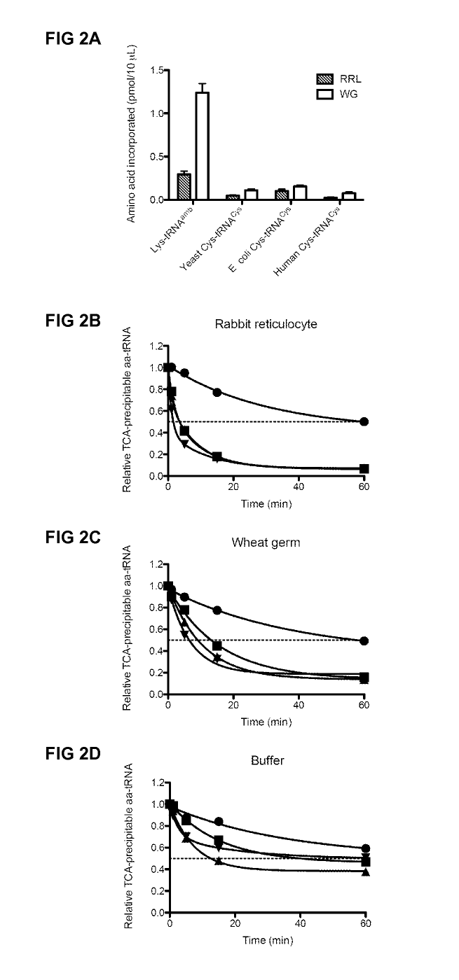

Resulting [¹⁴C]Cys-tRNAs were then purified and added to cell free translation reactions programmed with a truncated mRNA transcript containing a unique cysteine codon at residue 44 (FIG. 1). Hot trichloroacetic acid (TCA) precipitation of translation products revealed poor incorporation of [¹⁴C]Cys when compared to incorporation of [¹⁴C]Lys at an equivalent amber (UAG) codon using a control amber suppressor tRNA, [¹⁴C]Lys-tRNA^{amb} (FIG. 2A). One reason for this was that all three Cys-tRNAs underwent rapid deacylation (FIGS. 2B and 2C). Notably, >50% of [14C]Cys-tRNA^{cys} was deacylated within five minutes in RRL (FIG. 2B) and fifteen minutes in WG (FIG. 2C), whereas ˜50% of [¹⁴C]Lys-tRNA^{amb} remained intact after one hour (FIG. 2B, FIG. 2C and Table 2).

TABLE 2
Apparent half lives of Cys-tRNAs in RRL
and WG cell-free translation systems.

Apparent half-life^{a}
(min)

| aa-tRNA | RRL | WG |
|---|---|---|
| Unmodified aa-tRNAs | | |
| Lysine-tRNA^{amb} | 60.0 | 57.7 |
| Yeast Cys-tRNA^{Cys} | 3.5 | 9.1 |
| E. coli Cys-tRNA^{Cys} | 3.5 | 13.2 |
| Human Cys-tRNA^{Cys} | 1.6 | 6.5 |
| Yeast Cys-tRNA^{amb} | 1.6 | 13.4 |
| Yeast Cys-tRNA^{opl} | 2.5 | 20.5 |
| Yeast Cys-tRNA^{och} | 3.4 | 18.1 |
| E. coli Cys-tRNA^{opl} | 2.4 | 11.1 |
| Modified aa-tRNAs | | |
| εNBD-Lysine-tRNA^{amb} | >60 | >60 |
| Yeast NBD-Cys-tRNA^{Cys} | 10.8 | >60 |
| E. coli NBD-Cys-tRNA^{Cys} | 4.0 | >60 |
| Human NBD-Cys-tRNA^{Cys} | 2.3 | 54.1 |
| Yeast NBD-Cys-tRNA^{amb} | 12.0 | >60 |
| Yeast NBD-Cys-tRNA^{opl} | 13.6 | 58.9 |
| Yeast NBD-Cys-tRNA^{och} | 15.0 | >60 |
| E. coli NBD-Cys-tRNA^{opl} | 3.9 | >60 |
| Yeast MBB-Cys-tRNA^{opl} | 5.5 | 42.2 |

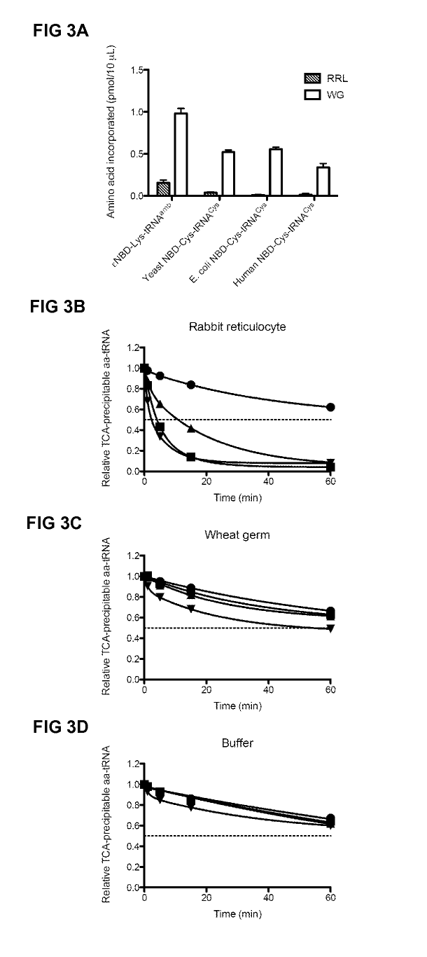

^{a}The apparent half live was determined by fitting the amount of cold-acid precipitable aa-tRNA as a function of time (FIG. 2B-D, FIG. 3B-D) to a one phase or two phase decay function, depending on a sum-of-squares F test (p < 0.05), and subsequently calculating at which time point during incubation half of the starting material was deaminoacylated.

Substantial deacylation was also observed under equivalent buffer conditions (FIG. 2D). Thus, synthetic Cys-tRNAs were both less chemically stable than their Lys counterparts, and more susceptible to enzymatic breakdown, possibly by aaRS trans-editing activity in WG and RRL.

To determine whether the cysteine side chain contributed to the poor stability of the aminoacyl-tRNA bond, a fluorescent moiety, 7-nitrobenz-2-oxa-1,3-diazole (NBD) was attached to the sulfhydryl and the ε-amino group of [14C]Cys-tRNAs and [¹⁴C]Lys-tRNA^{amb} using iodoacetamide or succinimide ester derivatives, respectively. NBD attachment markedly improved stability of the Cys-tRNAs in both WG and buffer (FIGS. 3C and 3D and Table 2), and correspondingly improved [¹⁴C]Cys incorporation efficiency in WG (FIG. 3A, compare to FIG. 2A). In RRL, however, with the exception of yeast NBD-[¹⁴C]Cys-tRNA^{cys} (FIG. 3B and Table 2), cysteine modification did not substantially change the rate of deacylation or [¹⁴C]Cys incorporation. Thus, Cys-tRNA deacylation activity in RRL is also able to recognize and remove the NBD-modified Cys residue. These studies demonstrate that cysteine modification enhances aa-tRNA stability and incorporation efficiency.

Next, the GCA anticodon in yeast and E. coli tRNA^{cys} was mutated to CUA, UCA, or UUA to generate tRNA^{amb(Cys)}, tRNA^{opl(Cys)}, and tRNA^{och(Cys)}, respectively. Converting the UGC anticodon to any of the suppressor anticodons nearly abolished aminoacylation in S-100 extract (Table 1). However, addition of purified recombinant human Cys-aaRS restored aminoacylation of yeast-derived tRNAs, while the addition of either E. coli or human Cys-aaRS restored aminoacylation of E. coli-derived tRNAs (Table 1).

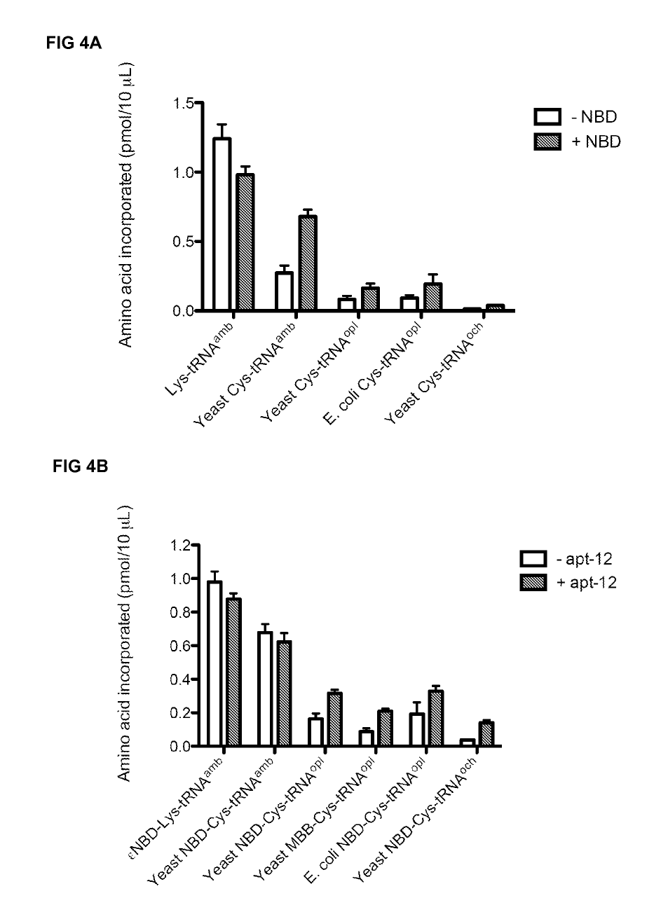

The stability of all suppressor Cys-tRNAs was similar to their WT counterparts in WG and RRL, both before and after NBD-labeling (Table 2). Nonetheless, despite its rapid deacylation, [¹⁴C]Cys-tRNA^{amb} suppressed termination at the UAG codon with ˜25% efficiency to that of [¹⁴C]Lys-tRNA^{amb} in the WG system, and the more stable NBD-[¹⁴C]Cys-tRNA^{amb} suppressed termination with 70% efficiency compared to εNBD-[¹⁴C]Lys-tRNA^{amb} (FIG. 4A). NBD-[¹⁴C]Cys-tRNA^{opl} and NBD-[¹⁴C]Cys-tRNA^{och} also incorporated the labeled probe at UGA and UAA codons, respectively. However, readthrough was significantly less efficient at opal and ochre codons that at the amber codon despite similar stabilities of NBD-labeled tRNAs, and similar suppressor aa-tRNA concentration in the translation reaction (Table 2 and FIGS. 4A and 4B).

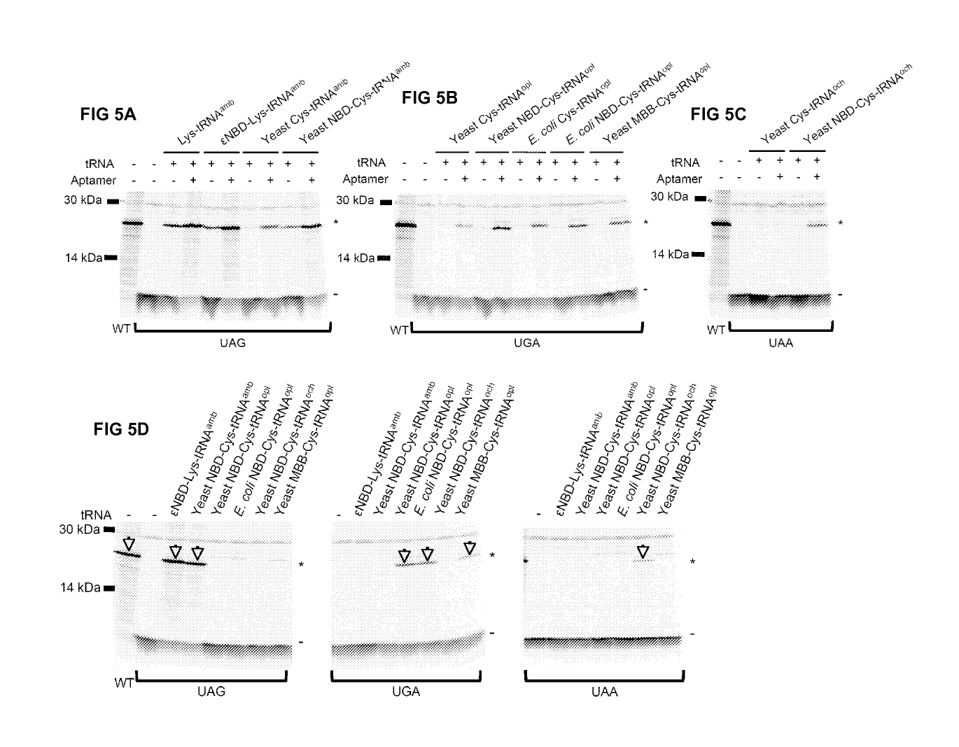

One explanation for the poor suppression of opal and ochre codons was that the corresponding tRNAs were less efficient than amber at competing with eukaryotic translation release factors (eRF1/eRF3). Because these synthetic tRNAs are identical in every aspect with the exception of the anticodon, it seems unlikely that they would exhibit different rates of eEF1-α GTP hydrolysis or a different induced conformational fit upon codon-anticodon base pairing at the ribosome A-site. Rather, the present results demonstrate that eukaryotic release factors, eRF1/eRF3, terminate translation more efficiently at opal and ochre as opposed to amber codons. Consistent with this hypothesis addition of an RNA aptamer previously shown to inhibit eRF1/eRF3 improved NBD-[¹⁴C]Cys incorporation efficiency by two-fold at the opal, and four-fold at the ochre, codon (FIG. 4B). Aptamer also improved translational readthrough in full-length constructs at all three stop codons when ³⁵S-labeled translation products were analyzed by SDS-PAGE (FIGS. 5A-5C). Moreover, eRF1/eRF3 inhibition stimulated readthrough of opal and ochre codons to a much greater extent than amber codons (FIGS. 5A-5C), and the aptamer concentration needed to achieve maximum readthrough was greater for opal than amber. The extent of readthrough augmentation by aptamer was more pronounced under these latter conditions due to differences in translating truncated versus full-length mRNA (discussed below).

Each of the three suppressor tRNAs exhibited a high degree of specificity, since translational readthrough was only observed when the cognate stop codon was present in the mRNA transcript (FIG. 5D). Moreover, when non-aminoacylated tRNAs were added to translation reactions at similar concentrations, stop codon readthrough was essentially undetectable, indicating that these suppressor tRNAs are not appreciably charged with endogenous amino acids in the translation reaction. Together, these results demonstrate that tRNA^{Cys}-derived suppressor tRNAs can be used to selectively incorporate non-natural amino acids at all three nonsense codons and that RNA aptamers directed against eRF1/eRF3 improve opal and ochre readthrough.

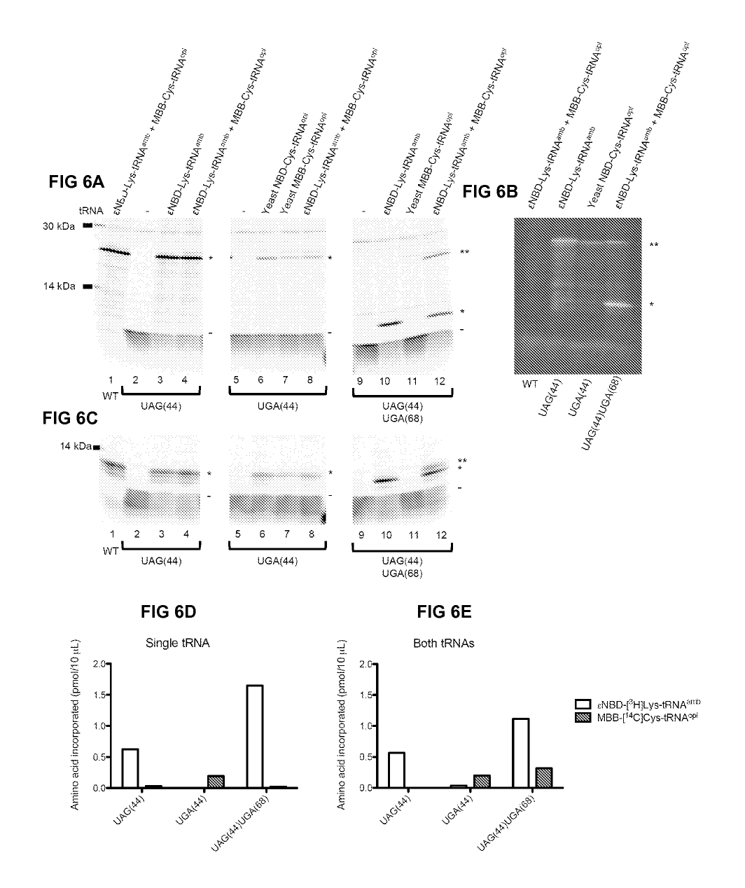

Next it was evaluated whether two different modified amino acids could be concurrently incorporated at two different nonsense codons in a single polypeptide. Yeast [¹⁴C]Cys-tRNA^{opl} was labeled with monobromobimane (MBB) [3-(bromomethyl)-2,5,6-trimethyl-1H,7H-pyrazolo(1,2-α)pyrazole-1,7-dione] and E. coli [³H]Lys-tRNA^{amb} was labeled with NBD. MBB-Cys-tRNA^{opl} demonstrated only slightly lower stability (Table 2) and slightly lower incorporation efficiency than NBD-Cys-tRNA^{opl} (FIG. 4B and FIG. 5B). Full length and truncated mRNAs containing UAG at codon 44 and/or UGA at codon 68 (FIG. 1) were translated in the presence of εNBD-Lys-tRNA^{amb}, MBB-Cys-tRNA^{opl}, or both tRNAs (FIG. 6). Quantitation of full-length protein by phosphorimaging revealed a cumulative readthrough efficiency at both stop codons of 16% when compared to the control (WT) protein (FIG. 6A). When gels were scanned for NBD fluorescence, a clear signal was detected whenever an NBD probe was incorporated (FIG. 6B). Fluorescent signal of MBB was too low to detect by in-gel scanning.

Further analysis revealed that the efficiency of stop codon readthrough was increased when the amber codon was followed by an opal codon, particularly in a truncated mRNA lacking a long 3′ untranslated region (FIGS. 6C-6E). This may have occurred because translation termination at the downstream opal codon allowed ribosomes to repeatedly reinitiate translation at the 5′ end of the mRNA and thereby increase the apparent readthrough efficiency at the amber codon. In fact, when protein bands were corrected for [³⁵S]-methionine content, the number of polypeptides that translated through the amber codon under these conditions actually exceeded the number of polypeptides produced from the WT protein (FIG. 6C, compare lane 1 to lane 10). Quantification of ε-NBD[³H]-Lys and MBB[¹⁴C]-Cys isotope incorporation also verified that readthrough at the upstream UAG codon by the single amber suppressor tRNA was increased three-fold when the downstream UGA codon was present (FIG. 6D) and approximately two-fold when translation was carried out in the presence of both amber and opal tRNAs. Thus, using complementary suppressor tRNAs carrying non-natural amino acids, together with inhibition of release factors eRF1/eRF3, a combined incorporation efficiency at two consecutive stop codons of 28% was achieved when compared to the parent protein (FIG. 6C, lanes 1 and 12).

In these studies, the inventors developed a generally applicable system for the site-specific incorporation of multiple probes into one protein. This approach is facile to implement, highly selective for the incorporation site, allows incorporation of diverse probes, and requires no other changes in protein sequence. Cys-tRNAs were excellent candidates to achieve these goals because the reactive sulfhydryl side chain can be conveniently modified with commercially available reagents under mild conditions that leave the labile aminoacyl bond intact. Although modified Cys-tRNA^{Cys} has previously been used to co-translationally incorporate a fluorescent NBD probe using a eukaryotic cell-free expression system, selective incorporation at the desired site required removal of endogenous native cysteine residues.

To overcome this limitation, the inventors developed tRNA^{Cys}-derived suppressor tRNAs that selectively suppress translation termination at their cognate stop codons. Radiolabeled and modified suppressor Cys-tRNAs were generated by enzymatic acylation of synthetic tRNA using a combination of E. coli extract and recombinant Cys-aaRS, followed by modification of the sulfhydryl moiety. These studies show that the native cysteine (GCA/ACA) anticodon presence was clearly not essential under the in vitro conditions used herein.

A requirement for co-translational probe incorporation is that the modified aa-tRNA species must remain stable during the time course of translation. Indeed, it was observed in the present studies that [¹⁴C]Cys-tRNA^{cys} and its suppressor derivatives exhibited low levels of translational incorporation due to rapid deacylation as determined by release of [¹⁴C]Cys from acid precipitable tRNA. However, modification of the sulfhydryl moiety with either of two different fluorescent dyes, MBB and NBD, markedly stabilized the aminoacyl bond and increased incorporation efficiency. Based on these observations, the instability of Cys-tRNAs seems to arise from both a chemical component, most likely caused by the reactive sulfhydryl side chain as demonstrated by spontaneous deacylation in buffer, and an enzymatic component, most probably due to trans-editing aaRS activity present in both RRL and WG. Cysteine modification markedly improved the chemical stability of Cys-tRNA and overcame trans-editing activity in WG but had little stabilizing effect in RRL. The contribution of aa-tRNA stability to stop codon suppression is therefore dependent upon both the nature of the attached aa moiety and pertinent deacylation enzymes present in the translation system.

NBD-Cys-tRNA^{amb} enabled translational readthrough in WG with an efficiency that approached εNBD-Lys-tRNA^{amb}. Thus both amber suppressor tRNAs compete effectively with eukaryotic release factors eRF1/eRF3 even though they derive from different tRNA scaffolds. Readthrough at opal and ochre codons, however, was markedly less efficient. A similar pattern obtained for orthogonal tRNA^{Gln}-based suppressors was previously attributed to differences in aminoacylation efficiency. In contrast, tRNA^{Cys}-derived suppressors used here are all present at equivalent concentrations, suggesting that reduced suppression at opal and ochre codons is more likely due to less efficient competition with translation termination factors eRF1/eRF3. One possible explanation is that Watson-Crick codon-anticodon base-pairing with amber tRNA in the ribosome A site more favorably stimulates the forward reaction of eEF-1α GTP hydrolysis and peptide bond formation. Weaker ternary interactions between mRNA, tRNA, and the ribosome might therefore lead to premature aa-tRNA release, possibly necessitating multiple tRNA entry events, each of which would presumably compete with release factors for translational readthrough. However, tRNA^{Cys(amb)}, tRNA^{Cys(opl)}, and tRNA^{Cys(och)} are identical in every aspect with the exception of the anticodon. Thus, unless the altered anticodon bases or subtle differences in their hydrogen bonding to amber, opal and ochre codons exert long range structural effects, these tRNAs would not be expected to exhibit major conformational differences at the ribosome decoding center.

If the rate of tRNA entry into the A site and subsequent induced fit are similar for otherwise matched tRNA^{Cys}-derived suppressors, differences in suppression efficiency more likely reflects in intrinsic binding of eRF1/eRF3 to amber, opal and ochre stop codons and/or subsequent conformational changes required for formation of the mature termination complex for a given binding cycle. The findings that inhibition of release factors by aptamer preferentially stimulates readthrough at opal and ochre codons to a greater extent than amber, support this notion and suggest that eRF1/eRF3 retain a hierarchy in stop codon recognition. This behavior resembles the selective stop codon reprogramming observed in ciliates even though all three codons are utilized for translation termination in higher eukaryotes. Given that little is known about the relative binding affinities and molecular details of how eRF1/eRF3 induce translation termination at amber, opal, and ochre codons, the set of tRNAs developed can be useful in deciphering mechanisms that underlie differences in suppression efficiency.

Finally, the concurrent incorporation efficiency obtained here using amber and opal tRNAs containing a modified amino acid was approximately ten times higher for truncated mRNA and six times higher for full length mRNA than has been previously observed for native amino acids in a eukaryotic expression system. This approaches clearly demonstrates the following: i) improved conditions for in vitro aminoacylation of modified tRNACys, ii) selective incorporation of non-native amino acids at all three cognate stop codons, iii) improved readthrough efficiency obtained by aptamer-induced inhibition of eukaryotic release factors, and iv) the ability to accurately quantify translation readthrough at both probe sites by ¹⁴C and ³H isotope incorporation.

This analysis demonstrates that translation termination at the second introduced (UGA) stop codon enables ribosomes to rapidly reinitiate translation, thereby increasing the number of chances to readthrough the first (UAG) codon. Remarkably, this ribosome “recycling” can actually increase the “apparent” UAG codon readthrough efficiency to greater than 100% (FIGS. 6C-6E) and by extension, increase the number of ribosomes that reach and readthrough the second (UGA) codon. Because the magnitude of this increase is dependent upon the time required to reach the second stop codon, concurrent readthrough will, to some extent, be affected by the distance between upstream and downstream stop codons. One consideration is whether similar mechanisms operate during sequential stop codon readthrough in intact cells. Readthrough efficiency was also increased for mRNA truncated after the final (endogenous) stop codon when compared to mRNA transcribed from supercoiled plasmid, suggesting that a long 3′ untranslated region interferes with translation reinitiation. Optimization of mRNA length, coding sequence length, and stop codon positions are therefore important considerations.

Development of tRNA^{Cys}-derived suppressor tRNAs now allows simultaneous incorporation of at least two non-natural amino acids in the same protein at efficiencies suitable for diverse biochemical and biophysical studies. The selective strategy described here provides a new technology to position multiple probes at defined locations in a protein with minimal other alteration of its natural sequence.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. An isolated modified suppressor tRNA, comprising:
a modified tRNA^{cys}, wherein the tRNA^{cys} has been modified so that an anticodon of the tRNA is complementary to a stop codon;
a cysteine amino acid residue covalently linked to the modified tRNA^{cys} by aminoacylation generating a chemically reactive sulfhydryl side chain; and
a detectable label covalently linked to the sulfhydryl side chain.

2. The isolated modified suppressor tRNA of claim 1, wherein the stop codon is an amber (UAG), an opal (UGA) or an ochre (UAA) stop codon.

3. The isolated modified suppressor tRNA of claim 1, wherein the detectable label functions as an aminoacyl-tRNA stabilizing molecule.

4. The isolated modified suppressor tRNA of claim 1, wherein the detectable label is a fluorescent group, a phosphorescent group, a photoaffinity label, or a photo-caged group, a crosslinking agent, a polymer, a cytotoxic molecule, a saccharide, a heavy metal-binding element, a spin label, a heavy atom, a redox group, an infrared probe, a keto group, an azide group, or an alkyne group.

5. The isolated modified suppressor tRNA of claim 4, wherein the fluorescent group is 7-nitrobenz-2-oxa-1,3-diazol (NBD) or 3-(bromomethyl)-2,5,6-trimethyl-1H,7H-pyrazolo (1,2-α)pyrazole-1,7-dione (MBB).

6. The isolated modified suppressor tRNA of claim 1, wherein the detectable label is covalently linked to the sulfhydryl side chain by an iodoacetamide and maleimide ester derivative.

7. The isolated modified suppressor tRNA of claim 1, wherein the modified tRNA is a eukaryotic or prokaryotic tRNA.

8. The isolated modified suppressor tRNA of claim 7, wherein the eukaryotic tRNA is a yeast tRNA or human tRNA.

9. The isolated modified suppressor tRNA of claim 7, wherein the prokaryotic tRNA is an E. coli tRNA.

10. A kit including at least one isolated modified suppressor tRNA of claim 1 and at least one RNA aptamer.

11. The kit of claim 10, wherein the at least one RNA aptamer is an RNA aptamer which suppresses one or more translation termination release factors.

12. The kit of claim 11, wherein the RNA aptamer suppresses translation termination release factor 1 (eRF1), translation termination release factor 3 (eRF3) or a combination thereof.

13. The kit of claim 10, wherein the at least one RNA aptamer is RNA aptamer 12 or RNA aptamer 34.

14. A method of incorporating at least one non-natural amino acid into a single polypeptide, comprising:
contacting a template mRNA capable of in vitro translation containing at least one amber (UAG), an opal (UGA) or an ochre (UAA) stop codon with at least one isolated modified suppressor tRNA of claim 1 and at least one RNA aptamer in a cell-free in vitro translation system capable of in vitro translation under conditions sufficient such that at least one non-natural amino acid is incorporated into the single polypeptide at the at the site of translation of the amber, opal or ochre codon.

15. The method of claim 14, wherein the method is a method of incorporating at least two non-natural amino acids into a single polypeptide wherein the template mRNA contains at least two amber, opal or ochre stop codons.

16. The method of claim 14, wherein the at least one RNA aptamer suppresses one or more translation termination release factors.

17. The method of claim 16, wherein the RNA aptamer suppresses translation termination release factor 1 (eRF1), translation termination release factor 3 (eRF3) or a combination thereof.

18. The method of claim 17, wherein the translation system is a Wheat Germ translation system.

19. The method of claim 17, wherein the translation system is a reticulocyte lysate translation system.

20. The method of claim 17, further comprising preparing the at least one isolated modified suppressor tRNAs of claim 1, wherein preparing the at least one isolated modified suppressor tRNAs of claim 1, comprises:
combining the modified suppressor tRNA^{cys} with cysteine, an E. coli extract and a purified recombinant aminoacyl-tRNA synthetase under conditions sufficient for the cysteine amino acid residue to be covalently linked to the modified tRNA to generate a modified suppressor tRNA with a cysteine including a chemically reactive sulfhydryl side chain; and
combining a detectable label with the modified suppressor tRNA so that the detectable label is covalently linked to the chemically reactive sulfhydryl side chain.


SEQUENCE LISTING (12 sequences)

SEQ ID NO: 1 - DNA, 74 bp, Escherichia coli
ggcgcgttaacaaagcggttatgtagcggattgcaaatccgtctagtccggttcgactccggaacgcgcctcca

SEQ ID NO: 2 - DNA, 74 bp, Escherichia coli
ggcgcgttaacaaagcggttatgtagcggattttaaatccgtctagtccggttcgactccggaacgcgcctcca

SEQ ID NO: 3 - DNA, 74 bp, Escherichia coli
ggcgcgttaacaaagcggttatgtagcggatttcaaatccgtctagtccggttcgactccggaacgcgcctcca

SEQ ID NO: 4 - DNA, 75 bp, Saccharomyces cerevisiae
gctcgtatggcgcagtggtagcgcagcagattgcaaatctgttggtccttagttcgatcctgagtgcgagctcca

SEQ ID NO: 5 - DNA, 75 bp, Saccharomyces cerevisiae
gctcgtatggcgcagtggtagcgcagcagattctaaatctgttggtccttagttcgatcctgagtgcgagctcca

SEQ ID NO: 6 - DNA, 75 bp, Saccharomyces cerevisiae
gctcgtatggcgcagtggtagcgcagcagatttcaaatctgttggtccttagttcgatcctgagtgcgagctcca

SEQ ID NO: 7 - DNA, 75 bp, Saccharomyces cerevisiae
gctcgtatggcgcagtggtagcgcagcagattttaaatctgttggtccttagttcgatcctgagtgcgagctcca

SEQ ID NO: 8 - DNA, 75 bp, Homo sapiens
gggggtatagctcaggggtagagcatttgactgcagatcaagaggtccctggttcgaatccaggtgccccctcca

SEQ ID NO: 9 - protein, 461 aa, Escherichia coli [sequence omitted]

SEQ ID NO: 10 - protein, 755 aa, Homo sapiens
MADSSGQQGKGRRVQPQWSPPAGTQPCRLHLYNSLTRNKEVFIPQDGKKVTWYCCGPTVYDASHMGHARSYISFDILRRVLKDYFKFDVFYCMNITDIDDKIIKRARQNHLFEQYREKRPEAAQLLEDVQAALKPFSVKLNETTDPDKKQMLERIQHAVQLATEPLEKAVQSRLTGEEVNSCVEVLLEEAKDLLSDWLDSTLGCDVTDNSIFSKLPKFWEGDFHRDMEALNVLPPDVLTRVSEYVPEIVNFVQKIVDNGYGYVSNGSVYFDTAKFASSEKHSYGKLVPEAVGDQKALQEGEGDLSISADRLSEKRSPNDFALWKASKPGEPSWPCPWGKGRPGWHIECSAMAGTLLGASMDIHGGGFDLRFPHHDNELAQSEAYFENDCWVRYFLHTGHLTIAGCKMSKSLKNFITIKDALKKHSARQLRLAFLMHSWKDTLDYSSNTMESALQYEKFLNEFFLNVKDILRAPVDITGQFEKWGEEEAELNKNFYDKKTAIHKALCDNVDTRTVMEEMRALVSQCNLYMAARKAVRKRPNQALLENIALYLTHMLKIFGAVEEDSSLGFPVGGPGTSLSLEATVMPYLQVLSEFREGVRKIAREQKVPEILQLSDALRDNILPELGVRFEDHEGLPTVVKLVDRNTLLKEREEKRRVEEEKRKKKEEAARRKQEQEAAKLAKMKIPPSEMFLSETDKYSKFDENVSMVCPHMTWRAKSSAKGKPRSRSSSRLRRSSTRNICRWPRMEASSEGAQD

SEQ ID NO: 11 - DNA, 77 bp, Artificial Sequence
  RNA aptamer 34
gggagctcagaataaacgctcaacatcaccgtacgccgggcaactggcgctgattcgacatgagacacggatcctgc

SEQ ID NO: 12 - DNA, 77 bp, Artificial Sequence
  RNA Aptamer 12
gggagctcagaataaacgctcaagtacctgaaaatgggaagcagagcgagcctttcgacatgagacacggatcctgc